(19) United States
(12) Patent Application Publication
(10) Pub. No.: US 2017/0199186 A1
(43) Pub. Date: Jul. 13, 2017

(54) METHODS OF USING CELLS ASSOCIATED WITH AUTOIMMUNE DISEASE

(71) Applicant: Singapore Health Services PTE LTD., Singapore (SG)

(72) Inventors: Salvatore Albani, Singapore (SG); Maura Rossetti, Singapore (SG); Roberto Spreafico, Singapore (SG)

(21) Appl. No.: 15/316,402

(22) PCT Filed: Jun. 8, 2015
(86) PCT No.: PCT/SG2015/050145

§ 371 Date: Dec. 5, 2016

(30) Foreign Application Priority Data
Jun. 6, 2014 (SG) 10201403024U

(51) Int. Cl. G01N 33/564; G01N 33/68; G01N 33/50

(52) U.S. Cl. CPC G01N 33/564; G01N 33/505; G01N 33/6893; G01N 2800/50; G01N 2800/52; G01N 2800/24

(57) ABSTRACT

The present invention relates to methods of diagnosing autoimmune disease, predicting suitable treatments of autoimmune disease or identifying autoantigens of autoimmune disease using a population of immunogenic HLA-DR+ T cells.

### FIELD OF INVENTION

The present invention relates to methods for diagnosing autoimmune disease, predicting suitable treatments of autoimmune disease or identifying autoantigens in a patient including determining the number of certain T cell subpopulations.

### BACKGROUND OF THE INVENTION

Autoimmune disease is an abnormal response of an adaptive immune response against substances and tissues normally present in the vertebrate possessing the adaptive immune response. Autoimmunity occurs when check points of peripheral tolerance, including suppression by CD4^{+FOXP}3⁺ T_{reg} cells [Buckner J H (2010) Nat Rev Immunol 10: 849-859], fail to delete or otherwise inactivate self-reactive clonotypes, which are found at basal levels even in the absence of disease [Danke et al. (2004). J Immunol 172: 5967-5972]. There are estimated to be more than 80 different types of autoimmune disease. Autoimmune disease can often be chronic, debilitating or even life threatening and is among the most poorly understood and poorly recognized of any category of illness. Even with guidelines such as Witebsky's postulates it is very difficult to classify a disease as an autoimmune disease and in most cases even more difficult to diagnose a patient with an autoimmune disease. Currently, for most autoimmune diseases there is no diagnostic marker and diagnosis is based on the presenting signs and symptoms as well as ruling out other causes of these signs and symptoms. It has been estimated that autoimmune disease is responsible for more than USD100 billion in direct healthcare cost annually in the US alone. A better understanding of the disease is needed, as well as better, more effective methods of diagnosis and/or treatment.

The treatment of autoimmune diseases is typically with immunosuppression to decrease the immune response. Examples include corticosteroids such as prednisone. Corticosteroids are known to cause osteoporosis and other side effects with extended use. Alternatively or additionally, non-steroid drugs such as azathioprine, cyclophosphamide, mycophenolate, sirolimus, tacrolimus, methotrexate, antibodies such as antibodies to tumor necrosis factor-alpha (TNF-α) or antibodies to certain cytokines may be used. Generally most of the current treatments are not effective in all cases and are known to have side effects with extended use. Despite the successful introduction of biologics such as TNF-α antibodies and beta interferon, therapy of autoimmune disease such as rheumatoid arthritis, juvenile idiopathic arthritis and multiple sclerosis is still ineffective in a sizable proportion of patients, who are exposed to a treatment they are bound to fail, with high social and economic costs. The treatment also lacks specificity, equally targeting detrimental autoimmune as well as beneficial responses, with obvious health hazards [Scott et al. (2010) Lancet 376: 1094-1108]. To minimize use of treatments in non-responders various cell markers have been suggested (US2012/0088678). The methods taught are complicated as there are so many markers involved on a wide range of different cells. A simpler and more direct method may encourage diagnostic or prognostic use in order to minimize detrimental use of treatments in non-responders or to determine appropriate treatment regimes.

Development of alternative treatments is needed, preferably those that are effective to non-responders to current treatment or treatments having fewer side effects. As the causes of most autoimmune diseases are unknown, targeted treatment is not possible. As a step to develop alternative treatments it would be helpful to identify any autoantigens instigating the autoimmune disease to enable targeting autoantigens to stop, block or hinder an autoimmune reaction.

The understanding of the pathogenesis of autoimmune diseases is hampered by the inability to properly identify, investigate and manipulate pathogenic cells, which frustrates the development of targeted therapeutic approaches. To properly understand and interfere with aberrant immune responses, it is essential to identify and thoroughly characterize the tiny fraction of antigen-specific T and/or B cells responsible for initiating and fueling the disease (Mallone, et al. 2011 Clin Dev Immunol 2011: 513210). This cannot be accomplished in most autoimmune diseases, where the inciting antigens are not known and epitope spreading further complicates the autoantigen landscape (Vanderlugt and Miller 2002 Nat Rev Immunol 2(2): 85-95). In addition, the site of immune autoreactivity—where most auto reactive T cells are located—is usually out of reach (as in multiple sclerosis), or requires invasive procedures (as in arthritis) (Dornmair, et al. 2009 Semin Immunopathol 31(4): 467-477). In the absence of an easy access to pathogenic cells in humans, most mechanistic work has been done in animal models. However, they do not sufficiently adhere to human disease, as they usually consist of an artificial vaccination against a single selfantigen (Asquith, et al. 2009 European journal of immunology 39(8): 2040-2044). The combination of animal and human research limitations has so far prevented a major break-through in understanding mechanisms of disease, or developing targeted diagnostic and therapeutic tools. Advances in this area are likely to impact on the field of autoimmune disease as a whole, as pathogenic pathways are believed to be shared among different autoimmune diseases (Goris and Liston 2012 Cold Spring Harb Perspect Biol 4(3)). The identification of an accessible source of pathogenic cells would not only boost understanding of disease pathogenesis, but also greatly facilitate the establishment of improved and targeted therapeutic protocols.

The lack of proper tools to identify, investigate and manipulate pathogenic T cells in humans hinders a full understanding of disease pathogenesis and the development of targeted therapies. Indeed, affected tissues—enriched in pathogenic cells—are inaccessible for most diseases; conversely, blood is easily available but offers limited insight for peripherally localized diseases. This is likely due to the strict compartmentalization of tissue-specific immune responses, which become highly diluted in the bloodstream. In addition, in many conditions, including most autoimmune diseases, the inciting antigens are unknown, which makes antigen-dependent strategies for identification of antigen-reactive T cells unfeasible.

To properly understand and interfere with aberrant immune responses, it is essential to identify and thoroughly characterize the tiny fraction of antigen-specific T and/or B cells responsible for initiating and fueling the disease. This cannot be accomplished in most autoimmune diseases, as usually the inciting antigens are not known and the site of immune autoreactivity is out of reach.

There is thus need in the art for improved methods that allow to overcome at least some of the existing drawbacks.

### SUMMARY OF THE INVENTION

The present invention is based on the inventors' identification of a small population of pathogenic-like CD4⁺ T cells recirculating from the synovium into the bloodstream and resistant to therapy in children affected by juvenile idiopathic arthritis (JIA). This identification of an accessible source of pathogenic T cells does not only boost understanding of disease pathogenesis, but also greatly facilitates the development of improved and targeted therapeutic protocols.

In a first aspect, the present invention thus relates to a method of determining the effectiveness of a therapeutic regimen in a patient afflicted by an autoimmune disease comprising;

a) obtaining a biological sample from the patient;

b) enriching CD4⁺ T cells expressing HLA-DR from the biological sample;

c) optionally expanding the CD4⁺ T cells expressing HLA-DR; and

d) determining the number of CD4⁺ T cells expressing HLA-DR, wherein an elevated number of CD4⁺ T cells expressing HLA-DR compared to a reference value indicates an increased likelihood that the patient is or will be failing therapy.

Another aspect of the invention relates to a method of determining a patients risk of developing an autoimmune disease comprising;

a) obtaining a biological sample from the patient;

b) enriching CD4⁺ T cells expressing HLA-DR from the biological sample;

c) optionally expanding the CD4⁺ T cells expressing HLA-DR; and

d) determining the number of CD4⁺ T cells expressing HLA-DR wherein an elevated number of CD4⁺ T cells expressing HLA-DR compared to a reference value indicates an increased likelihood the patient has or is at risk of developing an autoimmune disease.

Another aspect of the invention relates to a method for identifying autoantigens in a patient afflicted by an autoimmune disease comprising;

a) obtaining a biological sample from the patient;

b) enriching CD4⁺ T cells expressing HLA-DR from the biological sample

c) contacting the biological sample with a candidate autoantigen; and

e) determining the number of CD4⁺ T cells expressing HLA-DR, wherein an elevated number of CD4⁺ T cells expressing HLA-DR compared to a reference value indicates an increased likelihood that the candidate autoantigen is an autoantigen related to the autoimmune disease.

Other aspects of the invention will be apparent to a person skilled in the art with reference to the following drawings and description of various non-limiting embodiments.

### BRIEF DESCRIPTION OF THE DRAWINGS

In the following description, various embodiments of the invention are described with reference to the following drawings.

FIG. 1. HLA-DR+ T cells are more frequent in peripheral blood of JIA patients failing anti-TNF-based combination therapy. A-B. Immunophenotyping was performed on peripheral blood samples from JIA patients, collected before (T0) and after (Tend) anti-TNF-based combination therapy. T_{conv} were gated as CD14⁻CD3⁺CD4⁺CD25^{low/-} FOXP3⁻ (top row), while T_{reg} cells were gated as CD14⁻CD3⁺CD4⁺CD25⁺FOX3⁺ (bottom row) within total PBMCs. A representative staining (A) and summary statistics (B) of HLA-DR expression over time are shown. B. Vertical lines represent SEM. n=10-15 per group, per time point. *when p<0.05 (two-tailed unequal variance t-test).

FIG. 2. HLA-DR+ T_{conv} display an activated pro-inflammatory phenotype. A-G. Differential marker expression within gated HLA-DR⁺ and HLA-DR⁻ T_{conv} from NO ID patients at T0. Markers are grouped by biological function. Cytokine production was measured after PMA/ionomycin stimulation. Each line corresponds to a patient. MFI: median fluorescence intensity. *when p<0.05; **when p<0.01; ***when p<0.001; ****when p<0.0001; ns: not significant (two-tailed paired t-test).

FIG. 3. The TCR repertoire of circulating HLA-DR⁺ T_{conv} is enriched in clonotypes of synovial T cells. Next-generation sequencing of TCRβ CDR3 regions was performed on blood or synovial cells of NO ID patients. The overlap is reported as absolute numbers (top panels) or frequencies relative to blood samples (bottom panels). The number of unique amino acid (AA) sequences in each sample is indicated in italic (bottom panels). For comparisons to be fair, the size of the larger TCR repertoire (HLA-DR⁻) must be reduced to the size of the smaller (HLA-DR⁺) by repeated random sub-sampling, as detailed in Methods. poly-JIA: polyarticular JIA; oligo-JIA: oligoarticular JIA.

FIG. 4. The pro-inflammatory features of HLA-DR⁺ T_{conv} are exacerbated in clinical failures. A-B. Extensive phenotyping of HLA-DR⁺ and HLA-DR⁻ Tconv from JIA patients was performed across disease activities and time. A. Results (% of expression) were fed to a hierarchical clustering algorithm and color-coded upon row-wise normalization. n=5-10 per group. B. Differential marker expression within gated HLADR⁺ and HLA-DR⁻ T_{conv} between ID and NO ID patients at T0. n=6-10 per group. C. BCL2 expression in gated T_{conv} in healthy donors (HD). D. HLA-DR⁺ and HLA-DR⁻CD14⁻CD4⁺CD25^{low/-} T_{conv} were sorted from total PBMCs of HD and stimulated for 7 hours with anti-CD3/CD28-coated beads in the absence (left) or presence (right) of T_{reg} cells, sorted as CD14⁻CD4⁺CD25^{high}CD127^{low/-}. The percentage of suppression was calculated as reduction of CD154 expression in the presence of T_{reg} cells relative to the condition without T_{reg} cells. Each line corresponds to a HD. *when p<0.05; **when p<0.01; ***when p<0.001 (two-tailed unequal variance t-test).

FIG. 5. HLA-DR⁺ T_{reg} cells are bona fide activated T_{reg} cells. A. Frequency of total T_{reg} cells over time. Vertical lines represent SEM. n=10-13 per group, per time point. B. Differential Ki67 expression within gated T_{reg} cells between ID and NO ID patients. Vertical lines represent SEM. n=4-9 per group, per time point. C. Methylation profile of the TSDR region within the FOXP3 locus of sorted T_{reg} cells from representative males and females (ID or NO ID) out of 8 total patients analyzed. As a reference, the methylation profiles of cells from representative HD out of 6 are reported. Ex vivo T_{conv} are methylated regardless of gender, while T_{reg} cells are completely demethylated in males, but only partially demethylated in females due to X-inactivation. FOXP3⁺ T_{conv} were obtained by in vitro 3-day stimulation with anti-CD3/CD28-coated beads. Methylation values for each CpG were color-coded according to the legend. D. Differential marker expression within gated T_{reg} cells from NO ID patients at baseline. Each line corresponds to a patient. E. BCL2 expression within gated T_{reg} cells from HD. F. T_{conv} were sorted from total PBMCs of HD and stimulated with anti-CD3/CD28-coated beads for 7 hours in the presence of HLA-DR⁺ or HLA-DR⁻ T_{reg} cells. Suppression of CD154 expression relative to a no-T_{reg} cell control is reported. Each line corresponds to a HD. *when p<0.05; **when p<0.01; ***when p<0.001; ns: not significant (two-tailed paired t-test).

FIG. 6. HLA-DR does not endow T cells with antigen-presenting capabilities. T cell lines obtained by stimulating sorted HLA-DR⁻CD14⁻CD4⁺ T cells from HD for 7 days with anti-CD3/CD28-coated beads. A. Expression of HLA-DR and CD86 before (Day 0) and after (Day 7) culture. B. Antigen-presenting capacity of T cell lines (>50% HLA-DR⁺) in 5-day co-cultures with fresh autologous T_{conv} labeled with CFSE, in the presence or absence of SEB. As a control, allogeneic monocyte-derived dendritic cells activated with LPS were used. C. Calcium flux in Fluo-4-loaded T cells lines (>50% HLA-DR⁺). The activating stimulus (either cross-linking secondary antibody or ionomycin) was added at the time indicated by the arrow. A representative experiment out of 4 is shown throughout the figure.

FIG. 7. Lower TCR repertoire diversity in synovium than in blood of NO ID patients. TCRβ CDR3 sequencing was performed on HLA-DR⁻ blood or synovial samples, corresponding to 98% of CD4⁺ T cell repertoire. The Renyi diversity index for different values of alpha is reported. The parameter alpha modulates the Renyi index sensitivity to frequent or rare TCR species. A higher Renyi index means higher diversity. Because the Renyi index is consistently lower in synovial than in blood samples across all values of alpha, the diversity of its TCR repertoire is lower than in blood.

FIG. 8. Phenotypic characterization of HLA-DR⁺ T_{conv} in HD. A. Frequency of HLA-DR⁺ T_{conv} in a representative HD out of 7 analyzed. B-H. Differential marker expression within gated HLA-DR⁺ and HLA-DR⁻ T_{conv} from HD. Markers are grouped by biological function. Cytokine production was measured after PMA/ionomycin stimulation. Each line corresponds to a HD. MFI: median fluorescence intensity. *when p<0.05; **when p<0.01; ***when p<0.001; ****when p<0.0001; ns: not significant (two-tailed paired t-test).

FIG. 9. Phenotypic characterization of HLA-DR⁺ T_{reg} cells in NO ID patients at baseline. A-C. Differential marker expression within gated HLA-DR⁺ and HLA-DR⁻ T_{reg} cells from NO ID patients at T0. Markers are grouped by biological function. Each line corresponds to a patient. **when p<0.01; ***when p<0.001; ****when p<0.0001; ns: not significant (two-tailed paired t-test).

FIG. 10. Phenotypic characterization of HLA-DR⁺ T_{reg} cells in HD. A. Frequency of HLA-DR⁺ T_{reg} cells in a representative HD out of 7 analyzed. B. Methylation profile of the TSDR region within the FOXP3 gene of sorted HLA-DR⁺ and HLA-DR⁻⁰ T_{reg} cells from a representative male donor out of 3 analyzed. As a control, the methylation profile of total T_{conv} and T_{reg} cells is reported. Methylation values for each CpG within the TSDR were color-coded according to the legend. C-G. Differential marker expression within gated HLA-DR⁺ and HLA-DR⁻ T_{reg} cells from HD. Markers are grouped by biological function. Each line corresponds to a HD. *when p<0.05; **when p<0.01; ***when p<0.001; ****when p<0.0001; ns: not significant (two-tailed paired t test).

FIG. 11. Gating strategy for immunophenotyping of HLA-DR⁺ T cells. Total PBMCs were thawed and immediately stained with a viability dye, CD14, CD4, CD3, CD25, FOXP3 and HLA-DR, and gated as shown.

FIG. 12. Stability of HLA-DR upon PMA/ionomycin stimulation. Total PBMCs were recovered overnight in complete medium, then stained with CD4, CD3, CD25, FOXP3 and HLA-DR, either immediately or after 6-hour stimulation with PMA/ionomycin. A representative donor out of 4 is shown.

FIG. 13. TCR repertoire of circulating HLA-DR⁻ T_{conv} is unaffected by in vitro expansion. TCRβ CDR3 sequencing was performed on blood HLA-DR- T_{conv} either freshly sorted or upon 14-day in vitro expansion with anti-CD3/CD28. The figure shows the averaged results of three wells independently expanded from the same patient. A. TCRβ V gene usage of fresh or expanded HLA-DR⁻ T_{conv}. B. Frequency of amino acid (AA) TCR sequence overlap between fresh or expanded blood HLA-DR⁻ T_{conv} and synovial CD4⁺ T_{conv}.

FIG. 14. Unfractionated circulating T cells from JIA patients with active disease do not reflect synovial inflammation. A-D. Representative staining of synovial and peripheral blood samples of JIA patients with active disease, segregated by function. T cells were gated as CD14⁻CD3⁺CD4⁺CD25^{low/-} FOXP3⁻. E. Phenotypic results (% of expression for all markers except CD3, for which MFI was used) were fed to a unsupervised hierarchical clustering algorithm and color-coded upon row-wise normalization. n=8.

FIG. 15. HLA-DR is a good candidate for isolating synovial-like T cells from peripheral blood. A. Correlation (Pearson's R) among the indicated markers on circulating T cells of patients in active disease, color-coded according to the degree of correlation. n=33. T cell lines (>50% HLA-DR+) were obtained by stimulating sorted HLA-DR-CD14-CD4+ T cells from healthy controls for 7 days with anti-CD3/CD28-coated beads. A representative experiment out of 4 is shown. Pairwise correlations (Pearson's R) were calculated for the indicated markers (median fluorescence intensity (MFI) for CD3, % positive for all other markers) measured on circulating CD4+ T cells of patients with active juvenile idiopathic arthritis (JIA), and then colour-coded as indicated in the legend and re-ordered according to the aggregate level of correlation (first principal component). Larger circles indicate higher IRI. n=32. For each marker from panel A, the percentage of pairwise comparisons with all other markers from the panel ensuing in correlation (|R|≧0.7), or no correlation (|R|<0.7), is reported. B. Calcium flux in Fluo-4-loaded T cell lines. The activating stimulus (either cross-linking secondary antibody or ionomycin) was added at the time indicated by the arrow. C. Antigen-presenting capacity of T cell lines in 5-day co-cultures with fresh autologous T cells labeled with CFSE, in the presence or absence of SEB. As a control, allogeneic monocyte-derived dendritic cells activated with LPS were used.

FIG. 16. Circulating pathogenic-like lymphocytes (CPLs) from patients with active disease mirror the inflammatory synovial T cell signature. Phenotypic results (% of expression for all markers except CD3, for which MFI was used) within gated CPLs and the bulk of blood or synovial CD4⁺ T cells from JIA patients in active disease were fed to a hierarchical clustering algorithm and color-coded upon row-wise normalization. White indicates not measured. n=8 per group.

FIG. 17. CPLs are enriched in clonotypes of synovial T cells. Next-generation sequencing of TCRβ CDR3 regions was performed on blood or synovial T cells of patients in active disease. All data refer to in silico-translated amino acid sequences. AB. The Renyi diversity index for different values of α (A) and summary statistics at α=1 (B), indicated by the red arrow in panel A, are reported. Differential species diversity is substantiated only if the Renyi index is consistently greater (or lower) across all values of the α parameter. α>1, weights more abundant species, α<1, weights more rare species. C. The TCR repertoire distance was investigated by building dendrograms based on Chao-modified Jaccard index (top panel) or by estimating overlap after appropriate rarefaction (bottom panels). D. Summary statistics for the overlap data as measured at highest synovial coverage (dotted line in panel C). E, Summary of the TCR repertoire pairwise Chao distances between CPL or blood samples and their paired synovial sample. For A and C, each panel corresponds to a patient. poly-JIA: polyarticular JIA; oligo-JIA: extended oligoarticular JIA. **when p <0.01; ***when p <0.001; ns: not significant (two-tailed paired t-test).

FIG. 18. CPLs correlate with unresponsiveness to therapy and disease activity in both juvenile and adult autoimmune arthritis. A. CPL representation over time in blood CD4+ T cells from JIA patients. All patients were NO ID at T0, and were segregated in prospective ID and NO ID based on their clinical activity at Tend. Vertical lines represent SEM. n=10-15 per group, per time point. B. CPL representation in blood CD4+ T cells from RA patients, segregated by disease activity score (DAS-28 3) and compared to healthy control samples (HC). Each dot corresponds to a patient. low/rem: DAS<3.2; mod: DAS<5.1; high: DAS>5.1. *when p<0.05; **when p<0.01; ****when p<0.0001 (two-tailed unpaired t-test).

FIG. 19. CPLs are enriched in clonotypes of synovial T cells. Next-generation sequencing of TCRβ CDR3 regions was performed on blood or synovial T cells of NO ID patients. All data refer to nucleotide sequences. A-B. The Renyi diversity index for different values of α (A) and summary statistics at α=1 (B), indicated by the red arrow in panel A, are reported. Differential species diversity is substantiated only if the Renyi index is consistently greater (or lower) across all values of the α parameter. α>1, weights more abundant species, α<1, weights more rare species. C. The TCR repertoire distance was investigated by building dendrograms based on Chao-modified Jaccard index (top panel) or by estimating overlap after appropriate rarefaction (bottom panels). D. Summary statistics for the overlap data as measured at highest synovial coverage (dotted line in panel C). For A and C, each panel corresponds to a patient. poly-JIA: polyarticular JIA; oligo-JIA: extended oligoarticular JIA. **when p <0.01; ***when p <0.001; ns: not significant (two-tailed paired t-test).

FIG. 20. Circulating pathogenic-like lymphocytes (CPLs) from patients with active disease mirror the inflammatory synovial T cell signature.

### DETAILED DESCRIPTION

The inventors have surprisingly found that CD4⁺ T cells expressing HLA-DR represent a subgroup of T cells that are useful for diagnosing and monitoring treatment efficiency of autoimmune disease and for the identification of autoantigens.

Accordingly, a first aspect of the invention relates to a method of determining the effectiveness of a therapeutic regimen in a patient afflicted by an autoimmune disease comprising;

a) obtaining a biological sample from the patient;

b) enriching CD4⁺ T cells expressing HLA-DR from the biological sample;

c) optionally expanding the CD4⁺ T cells expressing HLA-DR; and

d) determining the number of CD4⁺ T cells expressing HLA-DR, wherein an elevated number of CD4⁺ T cells expressing HLA-DR compared to a reference value indicates an increased likelihood that the patient is or will be failing therapy.

The term “therapeutic regimen”, as used herein, refers to a plan for treating autoimmune disease. The therapeutic regimen can be any treatment known in the art for a specific autoimmune disease or it may be an experimental treatment that is postulated to treat the autoimmune disease in a similar manner to the known treatments. Possible treatment regimens may include immunosuppressive agents, anti-inflammatory agents, beta interferon, thyroid supplements, blood transfusion, antilogous stem cell transplants, Disease-modifying antirheumatic drugs (DMARD's), and Non-steroidal anti-inflammatory drugs (NSAID's). Examples of anti-inflammatory agents include corticosteroids such as prednisone, and NSAID's such as acetaminophen, opiates, diproqualone, ibuprofen, Cox-2 inhibitors. Examples of immunosuppressive agents include azathioprine, cyclophosphamide, mycophenolate, sirolimus, tacrolimus, methotrexate, ciclosporin, D-penicillamine, gold salts, hydroxychloroquine, leflunomide, minocycline, sulfasalazine, antibodies such as antibodies to tumor necrosis factor-alpha (TNF-α) or other biologics.

Biologics are medicinal products such as vaccine, blood or blood components, somatic cell therapy, gene therapy, tissue, recombinant proteins, living cells, therapeutic antibodies used to treat autoimmune disease. Examples of biologics used to treat autoimmune disease may include beta interferon, thyroid supplements, blood transfusion, antilogous stem cell transplants,adalimumab, enanercept, infleximab, certolizumab, golimumab, rituximab, abatacept, anakinra, tocilizumab, muronomab, abciximab, daclizumab, basilimab, omaliizumab, efalizumab, natalizumab, certolizumab pegol, usterkinumab, belimumab, clenoiximab, keliximab, priliximab, teneliximab, vapaliximab, ibalizumab, aselizumab, apolizumab, benralizumab, cedelizumab, eculizumab, epratuzumab, erlizumab, fontolizumab, mepolizumab, ocrelizumab, pascolizumab, pexelizumab, reslizumab, rontalizumab, rovelizumab, rupizumab, siplizumab, talizumab, teplizumab, tocilizumab, toralizumab, vedolizumab, or visillizumab.

In various embodiments the therapeutic regimen comprises administration of a biological agent, preferably an antibody. As used herein, the term “antibody” refers to any monoclonal antibody, polyclonal antibody, bifuctional fusion peptide or any similar constructs that are able to attach to a specific epitope and neutralise or stop its activity. In various embodiments the antibody inhibits TNFα. Examples of antibodies that inhibit TNFα include adalimumab, enanercept, infleximab, certolizumab, and golimumab. However, any antibody able to attach to TNFα and inhibit the TNFα is contemplated in these embodiments. In various embodiments the therapeutic regimen comprises administration of methotrexate and/or prednisolone. In various embodiments the therapeutic regimen comprises administration of an antibody that inhibits TNFα and/or methotrexate and/or prednisolone. In various embodiments the therapeutic regimen is for the treatment of juvenile idiopathic arthritis comprising an antibody that inhibits TNFα and/or methotrexate and/or prednisolone.

As used herein the term “patient” refers to any individual or organism with an adaptive immune response system. The patient may include any Gnathostomata or jawed vertebrate, preferably mammals, more preferably humans. In various embodiments the humans are juveniles aged between 0-15 years old. In various embodiments the patient may potentially be suffering from an autoimmune disease. In various embodiments the patient may have been diagnosed with an autoimmune disease based on signs and symptoms of the patient. In various embodiments the patient may be undergoing treatment for an autoimmune disease.

As used herein the term “autoimmune disease” may refer to any disease that is shown to be based on the existence and/or action of autoreactive cells. Autoimmune disease may include Hashimoto's thyroiditis, Graves' disease, Systemic lupus erythematosus, Sjogren's syndrome, Antiphospholipid syndrome-secondary, Primary biliary cirrhosis, Autoimmune hepatitis, Scleroderma, Rheumatoid arthritis, Antiphospholipid syndrome-primary, Autoimmune thrombocytopenic purpura (ITP), Multiple sclerosis, Myasthenia gravis, juvenile idiopathic arthritis, acute disseminated encephalomyelitis, Addison's disease, Agammaglobulinemia, Alopecia areata, Amyotrophic lateral sclerosis, Ankylosing spondylitis, Autoimmune cardiomyopathy, Autoimmune hemolyticanemia, Autoimmune inner ear disease, Autoimmune lymphoproliferative syndrome, Autoimmune peripheral neuropathy, Autoimmune pancreatitis, Autoimmune progesterone dermatitis, Autoimmune polyendocrine syndrome, Autoimmune thrombocytopenic purpura, Autoimmune urticaria, Autoimmune uveitis, Behcets disease, celiac disease, cold agglutinin disease, Crohn's disease, Dermatomyositis, Diabetes mellitus type I, Eosinophilic fasciitis, Gastrointestinal pemphigold, Good pastures syndrome, Guillain-Barr syndrome, Hashimoto's encephalopathy, mixed connective tissue disease, Morphea, Nacolepsy, pemphigus vulgaris, polymyositis, primary biliary cirrhosis, relapsing polychondritis, Psoriasis, Psoriatic arthritis, Rheumatic fever, Temporal arteritis, Transverse myelitis, Ulcerative colitis, undifferentiated connective tissue disease, vasculitis, Wegeners granulomatosis or any known or suspected autoimmune disease known in the art.

In various embodiments the autoimmune disease is selected from rheumatoid arthritis, juvenile idiopathic arthritis and multiple sclerosis. In various embodiments the autoimmune disease is rheumatoid arthritis. In various other embodiments the autoimmune disease is juvenile idiopathic arthritis. In various other embodiments the autoimmune disease is multiple sclerosis.

In various embodiments the CD4⁺ T cells expressing HLA-DR are conventional T cells (T_{conv}) or regulatory T cells (T_{reg}), preferably conventional T cells. Human leukocyte antigen D region (HLA-DR) proteins are class II MHC receptor proteins that are typically found on the surface of specialised antigen presenting cells (APCs). Specialised antigen presenting cells are primarily dendritic cells, macrophages and B cells, and B cells are the only cell group that expresses HLA-DR constitutively. HLA-DR is generally not expressed in CD4⁺ T cells, but rather CD4⁺ T cells expressing HLA-DR represent a specific, small subgroup that are associated with autoimmune disease.

As used herein “conventional T cells” also referred to as T_{conv}, are conventional T helper cells (Th cells), also referred to as naive T helper cells or also known as CD4⁺ T cells because they express the CD4 glycoprotein on their surface. Like all T cells, they express the T cell receptor CD3 complex. In various embodiments the CD4⁺ conventional T cells are HLA-DR⁺ CD14⁻CD4⁺CD25^{low/-} conventional T cells, preferably HLA-DR⁺ CD14⁻ CD4⁺CD25^{low/-}FoxP3⁻ conventional T cells. These cells express the CD4 but do not express CD14, they either express very low levels of CD25 or they do not express CD25 at all. As the inventors have found, a small subgroup of T helper cells surprisingly express HLA-DR and are involved with autoimmune diseases. Accordingly, the T cells of interest herein are HLA-DR expressing T cells. Optionally, the T cells do not express FoxP3. A subpopulation of the HLA-DR⁺ expressing T_{conv} cells include circulating pathogenic-like lymphocytes (CPLs) that expressed Ki67.

In various embodiments the CD4⁺ T cells expressing HLA-DR are HLA-DR⁺ CD14⁻CD4⁺CD25^{low/-} conventional T cells, preferably HLA-DR⁺ CD14⁻CD4⁺CD25^{low/-}FoxP3⁻ conventional T cells. Without been limited to any theory it is hypothesized that HLA-DR⁺ T_{conv} comprise autoimmunogenic T cells recirculating from the site of the auto immune reaction.

In various other embodiments the CD4⁺ T cells expressing HLA-DR are HLA-DR⁺ CD14⁻CD3⁺CD4⁺CD25⁺ regulatory T cells, preferably HLA-DR⁺ CD14⁻CD3⁺CD4⁺CD25⁺FoxP3⁺ regulatory T cells. These cells express the CD4, CD3 and CD 25 but do not express CD14, surprisingly the cells express HLA-DR, and optionally they express FoxP3. Without wishing to be limited to any theory, it is believed that activated HLA-DR⁺ T_{reg} cells expand in an effort to reduce inflammation.

As used herein “biological sample” refers to any sample taken from the patient as defined above. Examples of biological samples may include tissue, whole blood, plasma, Peripheral blood mononuclear cells (PBMCs) synovial fluid, isolated synovial fluid mononuclear cells (SFMCs) or cells from the patient. The biological samples should be obtained through known ethical procedures to extract and if required isolate the particular biological sample of interest. The biological samples can be used immediately as fresh samples or they may be stored first. When biological samples are stored, ideally they remain equivalent to freshly-collected sample. Such storage methods are known in the art. In various embodiments the biological sample is a body fluid sample, preferably a blood sample. In various embodiments the biological sample includes mononuclear cells such as PBMCs or SFMCs.

The CD4⁺ T cells expressing HLA-DR may be enriched or isolated from the biological sample using enrichment means known in the art such as antibody filtration, flow cytometry such as fluorescence-activated cell sorting (FACS) or magnetic bead sorting. Alternatively, any enrichment method known in the art would be suitable provided CD4⁺ T cells expressing HLA-DR are identifiable.

The CD4⁺ T cells expressing HLA-DR may optionally be expanded using methods known in the art. One such method is to stimulate the freshly enriched CD4⁺ T cells expressing HLA-DR with anti-CD3/CD28-coated beads, tetanus toxoid peptides, PMA/ionomycin or other antigens known to enhance T cell proliferation. The cells are preferably expanded in a medium that enables cell growth and proliferation. Many cell growth mediums suitable for this purpose as well as various expansion methods are known in the art.

The number of CD4⁺ T cells expressing HLA-DR may be determined using detection reagents, such as detectable antibodies that attach to CD4⁺ T cells expressing HLA-DR. Any suitable method known in the art able to quantify CD4⁺ T cells expressing HLA-DR in a sample would also be suitable. Preferred are the use of fluorescent antibodies and methods that include their use, such as FACS.

Surprisingly, the inventors observed a trend in patients undergoing autoimmune disease treatment, namely that the number of HLA-DR⁺ T cells declined in patients who responded to a given treatment regimen and increased in patients who did not respond to a given treatment regimen. Both HLA-DR⁺ T_{conv} and HLA-DR⁺ T_{reg} cells declined in patients who responded, and increased in patients who did not respond to a treatment regimen. This trend was distinctive to CD4⁺ T cells expressing HLA-DR. Based on this finding, it was concluded that an elevated number of CD4⁺ T cells expressing HLA-DR compared to a reference value indicates an increased likelihood that the patient is or will be failing therapy. On the other hand, a number equal to or lower than the reference value of CD4⁺ T cells expressing HLA-DR indicates an increased likelihood that the patient is responding to or will respond to the therapy. “Reference value”, as used herein, refers to a value that may reflect a normal undiseased state or individual or may reflect a reference point in a therapeutic regimen, such as the starting point prior to administration of any medication. The reference value depends on the actual method carried out and the object aimed at and may be easily determined by those skilled in the art by routine techniques.

“Elevated” or “increased” in relation to the number of T cells, as used herein, relates to a detectable increase compared to the reference value. Typically, such an increase means at least 10% compared to the reference value, preferably at least 20%, more preferably at least 50%. Similarly, “reduced” or “decreased” in relation to the number of T cells, as used herein, relates to a detectable decrease compared to the reference value. Typically, such a decrease means at least 10% compared to the reference value, preferably at least 20%, more preferably at least 50%.

In various embodiments the biological sample is obtained during the course of the treatment of the patient with the therapeutic regimen and an elevated number of T cells expressing HLA-DR compared to a reference value indicates an increased likelihood that the patient is failing therapy. This may provide the advantage of allowing identifying a non-responder, such as a patient that is failing therapy, and consequently stop the treatment to minimize any side effects from the non-beneficial therapy.

In various embodiments, a first biological sample is obtained prior to the therapeutic regimen and a second biological sample is obtained during the therapeutic regimen, wherein an elevated number of T cells expressing HLA-DR in the second sample compared to the first sample indicates an increased likelihood that the patient is failing therapy. Again, this may provide the advantage of allowing identifying a non-responder, such as a patient that is failing therapy, and consequently stop the treatment to minimize any side effects from the non-beneficial. therapy.

Similarly, where the number of T cells expressing HLA-DR in the second sample is similar to or reduced compared to the number of T cells expressing HLA-DR in the first sample, this indicates an increased likelihood that the patient is responding to therapy. This may provide the advantage of allowing identification of a responder, so that the therapeutic regimen may be continued with the expectation that the treatment regimen may be beneficial.

In various embodiments the method further comprises determining at least one additional marker expressed by the CD4⁺ T cells expressing HLA-DR, the at least one additional marker optionally being selected from the group consisting of Ki67, CCRS, CCR6, CTLA-4, LAG-3, PD-1, IL-4, IL-17, TNF-γ, and TNF-α. Alternatively, any markers known in the art that indicate the CD4⁺ T cells expressing HLA-DR is antigen-experienced would be suitable.

Another aspect of the invention relates to a method of determining a patient's risk of developing an autoimmune disease comprising;

a) obtaining a biological sample from the patient;

b) enriching CD4⁺ T cells expressing HLA-DR from the biological sample;

c) optionally expanding the CD4⁺ T cells expressing HLA-DR; and

d) determining the number of CD4⁺ T cells expressing HLA-DR, wherein an elevated number of CD4⁺ T cells expressing HLA-DR compared to a reference value indicates an increased likelihood the patient has or is at risk of developing an autoimmune disease.

While there are genetic risk factors that an individual may develop an autoimmune disease, there is no way to know if an offspring of an individual suffering from an autoimmune disease will also exhibit an autoimmune disease. It may be possible to screen such patients that may have an increased risk of developing an autoimmune disease with the described method. CD4⁺ T cells expressing HLA-DR can be used to define pathogenic clonotypes without prior knowledge of antigen specificity, a major unmet medical need in autoimmunity. Similarly, patients showing signs and symptoms of an autoimmune disease may be classified at being at risk of developing an autoimmune disease, in cases where an autoimmune disease has not (yet) been diagnosed by ruling out other causes of the signs and symptoms.

In various embodiments the CD4⁺ T cells expressing HLA-DR are conventional T cells or regulatory T cells, preferably conventional T cells, as described above. These cells may show the surface marker profile as has been disclosed in connection with the diagnostic methods of the invention above. The steps of obtaining a biological sample from a patient, enriching CD4⁺ T cells expressing HLA-DR from the biological sample and determining the amount of such cells that may optionally be expanded may be carried out as described in connection with the inventive methods above.

Another aspect of the invention relates to a method for identifying autoantigens in a patient afflicted by an autoimmune disease comprising;

a) obtaining a biological sample from the patient;

b) enriching CD4⁺ T cells expressing HLA-DR from the biological sample;

c) contacting the biological sample with a candidate autoantigen; and

e) determining the number of CD4⁺ T cells expressing HLA-DR, wherein an elevated number of CD4⁺ T cells expressing HLA-DR compared to a reference value indicates an increased likelihood that the suspected autoantigen is an autoantigen related to the autoimmune disease.

In various embodiments the CD4⁺ T cells expressing HLA-DR are conventional T cells or regulatory T cells, preferably conventional T cells, as described above. These cells may show the surface marker profile as has been disclosed in connection with the diagnostic methods of the invention above. The steps of obtaining a biological sample from a patient, enriching CD4⁺ T cells expressing HLA-DR from the biological sample and determining the amount of such cells that may optionally be expanded may be carried out as described in connection with the inventive methods above. In this connection, it is understood that any embodiment disclosed in the context of any one of the inventive methods is similarly applicable to the other methods of the invention.

The candidate autoantigen may be any substance normally present in a vertebrate possessing the adaptive immune response. In various embodiments the candidate autoantigen may be a substance suspected of being an autoantigen. Examples of candidate autoantigen may include peptides, proteins, heat shock proteins such as human 60 KDa Heat shock protein, p205, heterogeneous nuclear ribonucleoprotein A2 (RA33), filaggrin, carbohydrates, glycoproteins, fatty acids, cells, collagen, glycosylated type II collagen, cartilage, hyaluronic acid, lubricin, proteinases, collagenases, fibroblasts, or any other substances found anywhere in an individual with an adaptive immune response. Preferably, the candidate autoantigen is an epitope, such as a peptide or protein epitope. “Epitope”, as used herein, relates to a structure in a molecule recognized and bound by an antibody. In case the epitope is a peptide or protein epitope, it typically includes 6-12 amino acids.

In various embodiments the candidate autoantigen is isolated from a biological sample, preferably a synovial sample.

It should be understood that all embodiments disclosed above in relation to the methods of the invention, are similarly applicable to each method and use and vice versa.

As already described above, a population of circulating HLA-DR⁺CD4⁺ conventional T cells (T_{conv}) expanded in clinical failures to anti-TNF-based combination therapy enrolled in the Trial of early aggressive therapy in polyarticular juvenile idiopathic arthritis (TREAT trial) was identified. In the TREAT trial patients with juvenile idiopathic arthritis were given treatment with methotrexate, prednisone and an anti-TNF biologic (etanercept) and at the end of the treatment were categorized as responders where all signs and symptoms disappeared or non-responders where signs and symptoms remained at the end of the treatment [Wallace et al. (2012) ArthritisRheum 64: 2012-2021.]. These HLA-DR⁺CD4⁺ conventional T cells are highly activated, pro-inflammatory antigen-experienced cells recirculating from the inflamed tissue, and resistant to regulatory T cell (T_{reg} cell)-mediated suppression. Comparison of the TCR repertoire of blood HLA-DR⁺ T_{conv} and synovial CD4⁺ T cells demonstrated that HLA-DR⁺ T_{conv} are enriched in arthritogenic, potentially autoreactive clonotypes. Interestingly, HLA-DR⁺ T_{conv} increase in nonresponders is paralleled by an expansion of similarly activated HLA-DR⁺ T_{reg} cells, likely as a failed attempt to control inflammation. Based on the data, it is concluded that HLA-DR⁺ T cells provide a biological correlate for resistance to anti-TNF-based combination therapy and may be key contributors to the pathogenic autoimmune process. This provides an easily accessible source of pathogenic cells, which could be exploited for diagnostic tools and understand antigen specificity.

More generally, the present invention is based on the inventors' identification of a population of pathogenic T cells escaping from the site of autoimmune reaction that could be correlated with disease activity in both juvenile and adult autoimmune arthritis. These cells represent a circulating, easily accessible pool of pathogenic cells in an autoimmune disease, which can be used to substantially advance understanding of aberrant self-recognition as well as to design targeted diagnostic and therapeutic tools.

The finding that CPLs are enriched in pathogenic cells recirculating from the synovium is supported by their functional signature and, most importantly, their TCR repertoire. Indeed, CPLs are enriched in proliferating Ki67+ cells and preferentially express proinflammatory chemokine receptors and exhaustion markers, in the absence of overt infection. In addition, CPLs display lower levels of CD3 compared to the bulk of blood T cells, a sign of TCR-mediated activation, in contrast to cytokine-mediated bystander activation. However, the most direct evidence of CPL pathogenicity comes from the analysis of their TCR repertoire: indeed, CPLs are oligoclonal and share a consistent fraction of their TCR repertoire with the synovium, where arthritogenic T cells reside; this finding indicates that CPLs comprise pathogenic—and likely autoreactive—cells.

Although CPLs are present in ID patients and healthy controls, consistent with a basal level of self-reactive clonotypes even in the absence of disease, they are selectively expanded in NO ID patients and RA patients with highly active disease. Thus, CPLs provide a correlate and suggest a potential mechanism for unresponsiveness to therapy and disease activity. Indeed, CPL increase likely results from the expansion of arthritogenic suppression- and therapy-resistant T cells in the synovium, which boost synovial inflammation and thus foster the clinical manifestations of the disease.

Relying on a surrogate marker for CPL isolation embodies three major advancements. First, it bypasses the need for prior knowledge of antigen specificity, a major unmet medical need in autoimmune diseases such as JIA, where most autoantigens have not been identified. In addition, its effectiveness is not dependent on TCR affinity, a limitation currently imposed by tetramer-based strategies or restimulation approaches. Second, a surface protein devoid of signaling properties allows the isolation of (i) viable and (ii) unperturbed T cells, differently from other surrogate markers of antigen encounter (e.g. Ki67). Third, the long-lasting expression window of HLA-DR allows capturing a wide fraction of T cell emigrants from peripheral tissues, which would instead down-regulate early activation markers such as CD69 and CD40L by the time they reach the blood. This peculiar kinetics might be the reason why HLA-DR is the only activation marker tested that (i) is elevated in the inflamed synovium, and (ii) correlates with other pro-inflammatory molecules in the blood. It is demonstrated that each activation marker in the blood is unique in that it does not necessarily correlate with the T cell signature at the site of autoreactivity.

Although HLA-DR has been known as a T cell activation marker since the late '70s (Evans R L, et al. (1978) J Exp Med 148(5):1440-1445), it has been used to track antigen-specific clonotypes only for blood-borne infectious diseases, such as HIV (Smith MZ et al. (2013) BMC Infect Dis 13:100.). The use HLA-DR as a surrogate marker for detecting pathogenic T cells recirculating from peripheral tissues is proposed. Differently from blood-borne diseases, where it is easy to envision that pathogenic T cells can be easily isolated from blood, uncovering a small but detectable pool of circulating pathogenic T cells in an autoimmune disease is unexpected. Given the size of this pool, it is also easy to appreciate why investigations in whole blood for peripherally localized diseases have not been as fruitful. The isolation of pathogenic T cells through HLA-DR enables much more focused investigation; if followed by screenings with candidate self-peptides in non-pediatric settings, characterized by relaxed constraints on sample amount, might facilitate the definition of their TCR specificity, and might lead to the discovery of yet unknown autoantigens. The identification of the recirculating pathogenic T cells might especially useful for autoimmune diseases with difficult or no access to the actual site of inflammation

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples.

### EXAMPLES

In summary blood samples of JIA patients, before or after 6-12-month treatment with methotrexate, prednisone and an anti-TNF biologic (etanercept), were phenotyped to identify CD4⁺ T cell subsets correlating with responsiveness to therapy. HLA-DR⁺ conventional CD4⁺ T cells (Tconv) were expanded two-fold in clinical failures (p<0.05; n=10-15 per group, per time point). HLA-DR⁺ T_{conv} were analyzed for their phenotype, TCR repertoire, proliferation and resistance to suppression. Due to the limited sample amount withdrawable from children, 40 patients were allocated to different read-outs according to preliminary power calculations. HLA-DR⁺ T_{conv} were highly activated, pro-inflammatory antigen-experienced cells recirculating from the inflamed tissue, and resistant to regulatory T cell (T_{reg} cell)-mediated suppression. Comparison of the TCR repertoire of blood and synovial CD4⁺ T cells demonstrated that HLA-DR⁺ T_{conv} are enriched in arthritogenic clonotypes. Interestingly, HLA-DR⁺ T_{conv} increase in non-responders was paralleled by an expansion of similarly activated HLADR⁺ T_{reg} cells, likely as a failed attempt to control inflammation.

HLA-DR⁺ T cells provide a biological correlate for resistance to anti-TNF based combination therapy and constitute a reservoir of arthritogenic T cells recirculating from the inflamed synovium. This opens novel avenues of investigation to dissect the specificity of pathogenic TCRs for targeted clinical manipulation in JIA and other autoimmune diseases.

HLA-DR was identified as a marker of arthritogenic CD4⁺ T cells recirculating from the synovium into the bloodstream and resistant to anti-TNF-based combination therapy in children affected by juvenile idiopathic arthritis (JIA). This provides a valuable tool for investigating pathogenic immune processes and paves the way to more accurate and sustainable therapeutic approaches.

### Example 1

### Collection of Samples

Samples from 32 patients were collected from participants in the Trial of Early Aggressive Therapy in Polyarticular Juvenile Idiopathic Arthritis (TREAT) [Wallace et al. (2012) ArthritisRheum 64: 2012-2021.]. The TREAT study was IRB approved at all sites and all patients/parents signed assent/consent form for participation in this study and blood draw. The participants were naive to biologics at baseline and were treated with methotrexate (MTX, 0.5 mg/kg/week SQ, max 40 mg)+etanercept (ETN, 0.8 mg/kg/week SQ, max 50 mg)+prednisolone (0.5 mg/kg/day). The prednisolone was tapered to zero in 4 months. Blood was drawn before starting ETN (T₀) or after at least six months of treatment (T_{end}). Additional samples from 3 polyarticular and 5 extended oligoarticular JIA patients treated for at least 6 months with MTX+ETN were collected at G. Gaslini Institute, Genoa, and IRCCS Policlinico S. Matteo Foundation Pavia (Italy). Synovial fluid from joint aspirations performed on two NO ID patients to relieve swelling was processed to isolate synovial fluid mononuclear cells (SFMCs). Blood samples from 14 adult healthy donors (HD) were collected at the TSRI Normal Blood Donor Service (La Jolla, Calif.).

Blood and synovial fluid from 14 JIA patients with active disease were collected at G. Gaslini Institute, Genoa, and IRCCS Policlinico S. Matteo Foundation, Pavia (Italy). Blood from 39 RA patients was collected as part of the DNAJP1 study.

All samples were collected upon informed consent. All experiments have been conducted according to the principles expressed in the Declaration of Helsinki and approved by SBMRI IRB.

### Example 2

### Isolation and Cryopreservation of PBMCs

EDTA-anticoagulated blood was received and processed within 24 hours from withdrawal. Synovial fluid was processed immediately after withdrawal. Peripheral blood mononuclear cells (PBMCs) and SFMCs were separated by density gradient with Histopaque-1077 (Sigma-Aldrich) and frozen in freezing medium (90% FCS, 10% DMSO).

### Example 3

### Cell Culture

For fresh cells, complete medium was prepared as follows: RPMI-1640 (HyClone), 5% heat-inactivated human serum AB (GemCell), 2 mM L-Glutamine (Gibco) and 100 U/ml penicillin/100 μg/ml streptomycin (Gibco). To generate and expand T cell lines from HD, 10% FBS replaced human serum in the medium. T cell lines were established by stimulating freshly sorted HLA-DR⁻CD14⁻CD4⁺ T cells with anti-CD3/CD28-coated beads (Life Technologies) at a 1:5 ratio. Complete medium containing 20 U/ml rhlL-2 (Life Technologies) was added every 2-3 days. After 7 days, dead cells were removed with the Dead Cell Removal kit (Miltenyi Biotec) and viable cells were rested for 40 hours in complete medium. Before experiments, T cell lines were routinely tested to confirm that at least 50% of cells were positive for HLA-DR.

### Example 4

### Flow Cytometry

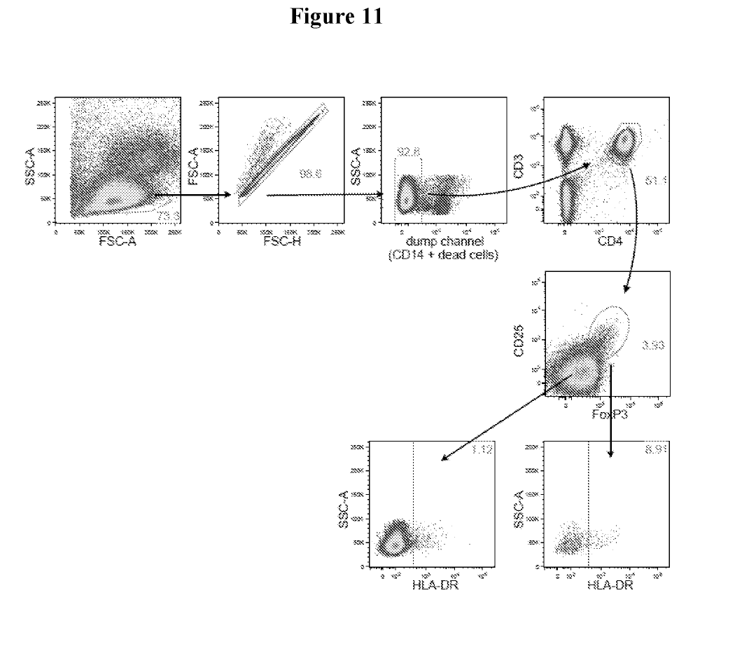

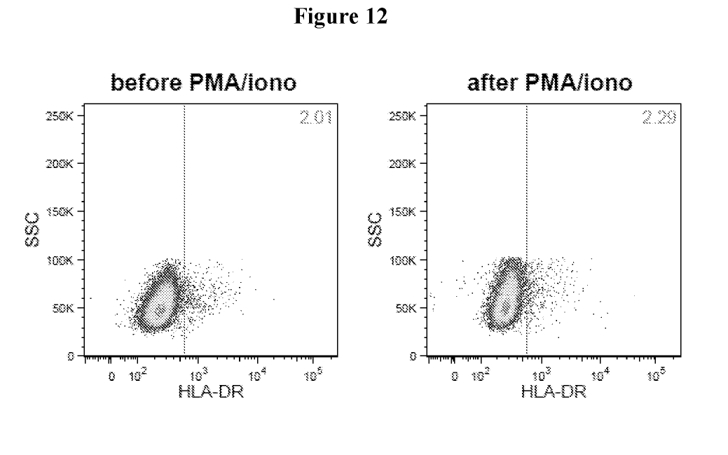

Ex vivo immunophenotyping was performed on PBMCs right after thawing. Dead cells were excluded using Live/Dead Fixable Near-IR Stain (Life Technologies). Fc receptors (FcR) were blocked using FcR blocking reagent (Miltenyi Biotec) to avoid non-specific antibody binding. Intracellular staining was performed with the FOXP3 buffer set (eBioscience). To measure cytokine production, total PBMCs were thawed and allowed to rest overnight in complete medium before stimulation with 20 nM PMA and 200 nM ionomycin (Fisher) for 6 hours. GolgiPlug and GolgiStop (BD Biosciences) were added to cell culture 2 hours after PMA/ionomycin stimulation. Samples were acquired with an LSRFortessa (BD Biosciences). Markers were measured on CD14⁻CD3⁺CD4⁺CD25⁺FOXP3⁺ T_{reg} cells and CD14⁻CD3⁺CD4⁺CD25^{low/-} FOXP3⁻ T_{conv} cells (FIG. 11). Antibodies were from Biolegend, BD Biosciences and eBioscience. Analysis was performed with FlowJo (Treestar). The stability of HLA-DR expression upon PMA/ionomycin stimulation is shown in FIG. 12.

### Example 5

### T Cell Proliferation and Suppression Assays

In pilot experiments, a remarkably different viability of HLA-DR⁺ and HLA-DR⁻ T cells was found after 5-day in vitro stimulation. To minimize the confounding effect of the differential viability upon culture, a short 7-hour suppression protocol [Canavan et al. (2012) Blood 119: e57-66] was used. Briefly, PBMCs were thawed and rested overnight in complete medium with 20 IU/ml rhIL-2. CD4⁺ T cells were enriched with the Human CD4⁺ T cell Isolation kit II (Miltenyi Biotec) and sorted in HLA-DR⁺ or HLA-DR⁻CD14⁻CD4⁺CD25^{low/-} T_{conv} and CD14⁻CD4⁺CD25^{high}CD127^{low/-} T_{reg} cells. To discriminate T_{conv} and T_{reg} cells at the end of the co-culture, either population was labeled with 0.02 μM CFDA-SE (Life Technologies). 5,000 T_{conv} and 5,000 T_{reg} cells were then stimulated with anti-CD3/CD28-coated beads (T_{conv}:bead=5:1) in the presence of fluorochrome-conjugated anti-CD154. At the end of the incubation, cells were stained with propidium iodide (PI, BioLegend) and acquired with a FACSAria II.

### Example 6

### TCR Repertoire

TCRβ CDR3 sequencing was performed by Adaptive Technologies [Robins et al. (2010) SciTransl Med 2: 47ra64.]. Dead cells were excluded using Live/Dead Fixable Near-IR Stain (Life Technologies) or PI. SFMCs were sorted in HLA-DR-positive and -negative CD14⁻CD3⁺CD4⁺CD25^{low/-}FOXP3⁻ T_{conv} (patient 1, poly-JIA) or HLA-DR-positive and -negative CD14⁻CD3⁺CD4⁺ T cells (patient 2, oligo-JIA) immediately after thawing. PBMCs were sorted in HLA-DR-positive and -negative CD14⁻CD3⁺CD4⁺CD25^{low/-} T_{conv} immediately after thawing. Genomic (gDNA) was extracted using the FFPE DNA MiniPrep kit (Zymo Research) from fixed cells, or the Agencourt Genfind v2 kit (Beckman-Coulter) from unfixed cells.

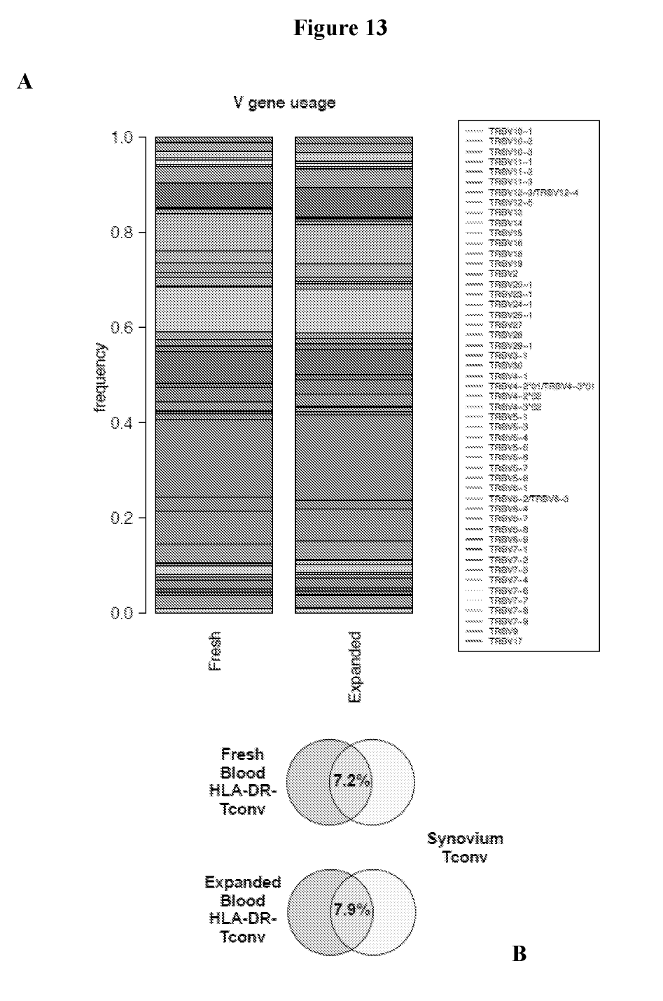

Because the number of HLA-DR⁺ T_{conv} did not meet the minimum sample requirement for TCRβ CDR3 sequencing, HLA-DR⁺ T_{conv} were expanded in vitro for 14 days according to an established protocol [Geiger et al. [(2009). J Exp Med 206: 1525-1534.] and the current results independently demonstrate that this protocol preserves the original relative frequencies of TCR species; indeed, similar results from fresh vs expanded HLA-DR⁻ T_{conv} was obtained in terms of TCRβ V gene usage and overlap between blood and synovial repertoires (FIG. 13). Nonetheless, to exclude any potential bias, a conservative analytical approach was opted for, by considering only presence/absence of HLA-DR⁺ T_{conv} TCR sequences, rather than counts and frequencies.

The analysis of TCR species composition is conceptually identical to the ecological investigation of biodiversity. Since ecological methods are well developed, they have been adapted to TCR repertoire analysis [Robins et al. (2010)]. For comparisons to be fair, the size of the larger TCR repertoire must be reduced to the size of the smaller by repeated random subsampling [Venturi et al. (2007) Journal of immunological methods 321: 182-195]. Because HLA-DR T_{conv} are more abundant than their HLA-DR⁺ counterparts, HLA-DR repertoires were subsampled by performing 1,000 draws without replacement. For each random draw, a number of unique TCR sequences equal to the number of unique TCR sequences of its paired HLA-DR⁺ sample was taken, weighted according to the relative frequencies of individual HLA-DR⁻ TCR species. For each blood sample (either the single HLA-DR⁺ T_{conv} or a random draw from HLA-DR⁻ T_{conv}), the number and fraction of unique amino acid TCR CDR3 sequences shared with its paired synovial sample was calculated. Amino acid sequences were favoured over nucleotide sequences because it is the former that ultimately determines TCR specificity. The median of results from subsamples was used as the aggregate index for the whole HLA-DR sample.

In order to quantitate the TCR repertoire diversity, the common Renyi entropy index was used [Rempala and, Seweryn (2012) Journal of mathematical biology; Cebula A, et al. (2013) Nature 497(7448):258-262.]. Diversity indices capture both species richness and distribution evenness. The larger the Renyi index, the larger the diversity. Because species frequencies are needed to compute the Renyi index, only the diversity of blood HLA-DR (˜98% Tconv) and synovial samples was compared. The Renyi index is modulated by the α parameter: a value of α above unity puts more weight on abundant species, while a value of α below unity puts more weight on rare species. Because, by definition, sampling of an entire population cannot be achieved, some TCR species will never be observed in the sample. Such undersampling mostly affects rare TCRs. Therefore, it is standard practice to evaluate the Renyi index across different values of α [Rempala and, Seweryn (2012)]. When comparing two samples, differential species diversity is substantiated only if the Renyi index is consistently greater (or lower) across all values of the α parameter. Similarly to other analyses of TCR repertoire, fair comparisons between Renyi indices require similar sample size. As such, larger samples were reduced to the size of their paired smaller sample by repeated (200 times) random subsampling. The median of the indices calculated for each random subsample was then plotted and used as the Renyi index of the whole sample [Venturi et al. (2007)].

### Example 7

### TSDR Methylation

HLA-DR-positive and -negative PI⁻CD14⁻CD3⁺CD4⁺CD25^{high}CD127^{low/-} T_{reg} cells were sorted from PBMCs right after thawing. gDNA was isolated with the ZR-Duet DNA/RNA MiniPrep kit (Zymo Research). Bisulphite conversion was performed with EZ DNA Methylation-Direct kit (Zymo Research). The TSDR of the FOXP3 locus was amplified with the Taq PCR core kit (Qiagen) using published primers [Baron et al. (2007) Eur J Immunol 37: 2378-2389]. The PCR product was purified with the MinElute PCR Purification Kit (Qiagen) and sequenced on a 3100 Genetic Analyzer (Applied Biosystems). Electropherograms were analyzed with ESME [Lewin et al. (2004) Bioinformatics 20: 3005-3012].

### Example 8

### Mixed Lymphocyte Reaction

CD14⁻CD4⁺CD25^{low/-} T_{conv} were sorted from thawed PBMCs after resting overnight in complete medium with 20 IU/ml rhlL-2. T_{conv} were labeled with Cell Trace Violet (Life Technologies). Allogeneic HLA-DR expressing T cell lines were labeled with CFDA-SE and used to stimulate sorted T_{conv} at 1:1 ratio in the presence of rhlL-2 (50 IU/ml) and, where indicated, SEB (10 ng/ml, Sigma-Aldrich). As a control, allogeneic monocyte-derived dendritic cells activated with LPS (50 ng/ml, Sigma-Aldrich) were added to fresh T_{conv} at 1:1 ratio. Dendritic cells were generated by incubating CD14⁺ monocytes in complete medium with rhGM-CSF (50 ng/ml) and rhlL-4 (10 ng/ml, both cytokines from R&D Systems) for 6 days.

### Example 9

### Calcium Flux

HLA-DR-expressing T cell lines were loaded with 2 μM Fluo-4 AM (Life Technologies) for 30 min at RT, then placed in Tyrode's solution containing Sytox Red (Life Technologies) as a viability dye. Cells were pre-warmed at 37 degrees before analysis. Calcium flux was measured at 37 degrees for 30 seconds (baseline)+1 min (addition of mouse primary Ab, 8 μg/ml)+5 min (addition of anti-mouse IgM/G, 16 μg/ml). Ionomycin was used as a positive control at 2μg/ml.

### Example 10

### Statistical Analyses

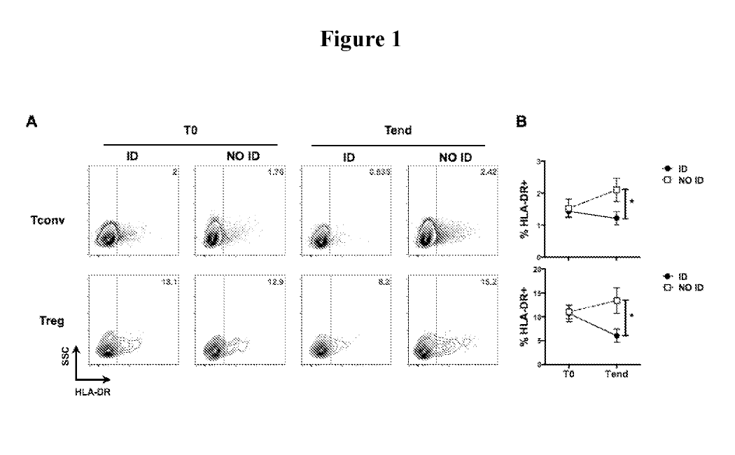

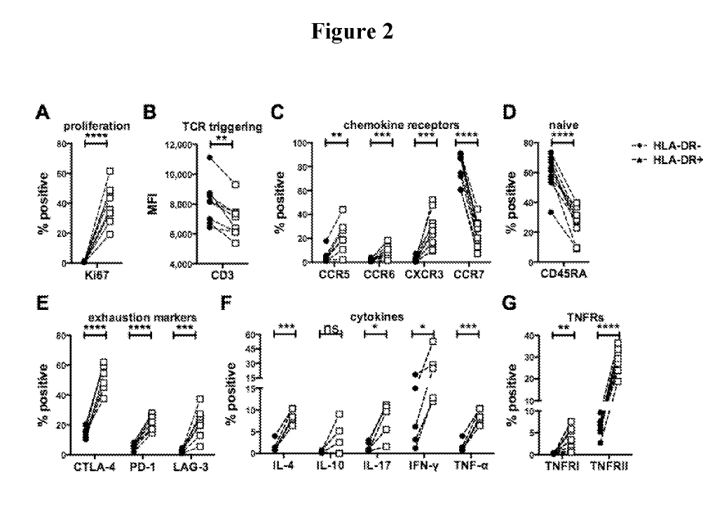

Due to limitations in pediatric sample amounts, the 40 patients were allocated to different read-outs based on power calculations. Pilot immunophenotyping of T cells revealed an effect size d=1.52 (T.) or d=2.39 (T_{reg}) for HLA-DR at T_{end} when comparing ID vs NO ID patients. With this effect size, the sample size needed to achieve 95% power at α=0.05 is 13 (T_{conv}) or 6 (T_{reg}) per group. 15 samples were used per group at T_{end} (FIG. 1B). For matched HLA-DR⁺ vs HLA-DR⁻ comparisons, preliminary experiments on normal donors suggested a large effect size (dz>2.5) for most markers. Even if only 4 samples per group were required to achieve 95% power with this effect size, most experiments were performed with higher n, as indicated in figure legends. Post-hoc analysis of patients' data (FIGS. 2, 4 and 5) confirmed the large effect size observed in pilot experiments on HLA-DR⁺ and HLA-DR⁻ T cells from HD.

Systematic visual inspection of plotted data indicated normal distributions with no prior need for transformation, supporting the use of two-tailed t-tests in comparisons. Paired tests were used in HLA-DR⁺ vs HLA-DR⁻ and matched CPLs vs blood vs synovium comparisons, while unequal variance unpaired tests were used in ID vs NO ID comparisons and in all other comparisons.

Hierarchical clustering was performed on phenotypic data using the euclidian distance and complete linkage.

### Example 11

### HLA-DR⁺ T Cells are More Frequent in Peripheral Blood of JIA Patients Failing Anti-TNF-Based Combination Therapy

To characterize the immunological mechanisms underlying responsiveness to therapy in JIA, peripheral blood samples from JIA patients, collected before (T₀) and after (T_{end}) an aggressive therapeutic regimen including methotrexate, prednisolone and etanercept (decoy TNF receptor II, TNFRII) were immunophenotyped. Patients were stratified by clinical activity based on whether they reached inactive disease (ID) [ Wallace C A, Ruperto N, Giannini E (2004) J Rheumatol 31: 2290-2294.] or not (NO ID) at T_{end}. Using flow cytometry, CD4⁺ T cells were inspected, a major player in autoimmune diseases, for differentiation (CD45RA, CCR7, CD62L, CD27), activation (CD30, CD69, GITR, HLA-DR, ICOS) and exhaustion (CTLA-4, PD-1). In addition, the expression of effector (Granzyme A and B) and regulatory (CD39, CD103, GARP) molecules were examined. To find potential mediators of pathogenesis, cell subsets mirroring clinical responsiveness, i.e. equally represented in ID and NO ID patients at baseline, but diverging over the course of therapy was the focus. In addition, because the decision to respond to antigens, including self-antigens, comes from the balance between the inflammatory and tolerogenic properties of T_{conv} and T_{reg} cells, respectively, dysregulation in both compartments was examined.

A trend was found whereby HLA-DR⁺ T cells uniquely fitted the parameter requirements. Both HLA-DR⁺ T_{conv} and HLA-DR⁺ T_{reg} cells declined in ID patients, they increased in NO ID patients. As a result, the HLA-DR⁺ T_{conv} and T_{reg} cell compartments of NO ID patients at T_{end} were both double in size than in ID patients (FIG. 1A-B). This trend was distinctive of HLA-DR; indeed, differences in other activation markers were either small/nil or inconsistent between T_{conv} and T_{reg} cells.

Based on these results, it can be assumed that HLA-DR⁺ T_{conv} cells comprise arthritogenic T cells recirculating from the synovium, and that their increase in the circulation of NO ID patients mirrors the increased synovial inflammation eventually leading to treatment failure; consequently, activated HLA-DR⁺ T_{reg} cells would also expand in an effort to reduce inflammation.

### Example 12

### HLA-DR⁺ T_{conv} Display an Activated Pro-Inflammatory Phenotype

To investigate the pathogenic potential of HLA-DR⁺ T_{conv} in NO ID patients, this subset was characterized at phenotypic and functional level. A large fraction of HLA-DR⁺ T_{conv} expressed Ki67, in contrast to HLA-DR⁻ T_{conv} (FIG. 2A). Moreover, HLA-DR⁺ T_{conv} downregulated CD3, a sign of TCR-mediated antigen recognition (FIG. 2B) [Bangs et al. (2009) J Immunol 182: 1962-1971]. Therefore, in the absence of overt infection, it is reasonable to expect that a consistent fraction of Ki67⁺CD3^{low} T_{conv} would be specific for self-antigens. Importantly, HLA-DR⁺ T_{conv} might have indeed been exposed to autoantigens in affected joints, as they preferentially expressed chemokine receptors directing cells to inflamed tissues (CCR5, CCR6 and CXCR3; FIG. 2C). Consistently, HLA-DR⁺ T_{conv} contained a lower proportion of lymphoid-homing CCR7⁺ (FIG. 2C) and naive CD45RA⁺ cells (FIG. 2D). Moreover, HLA-DR⁺ T_{conv} expressed higher levels of exhaustion markers, such as CTLA-4, LAG-3 and PD-1 (FIG. 2E), indicating that they are at a late stage of activation, again consistent with prolonged antigen exposure. In line with a primed phenotype, HLA-DR⁺ T_{conv} also showed higher production of IL-4, IL-17, IFN-γ and TNF-α (FIG. 2F).

The higher expression of TNF-α is compelling in light of the pivotal role of this cytokine in autoimmune arthritis [Scott et al. (2010)]. Nowadays, most JIA patients—including the subjects of this study—are treated with a combination therapy comprising anti-TNF. However, since the sensitivity to TNF-α depends on the expression of TNFRs, which is not uniform, TNF neutralization may not equally affect all T cells. To investigate the potential sensitivity of HLA-DR⁺ T_{conv} to anti-TNF therapy, TNFR expression was measured. Strikingly, a much larger proportion of these cells was positive for TNFRI and/or TNFRII compared to HLA-DR⁻ T_{conv} (FIG. 2G).

In summary, HLA-DR marks a population of antigen-experienced T_{conv} recirculating through inflamed sites and potentially affected by the TNF/anti-TNF balance.

### Example 13

### The TCR Repertoire of Circulating HLA-DR⁺ T_{conv} is Enriched in Clonotypes of Synovial T Cells

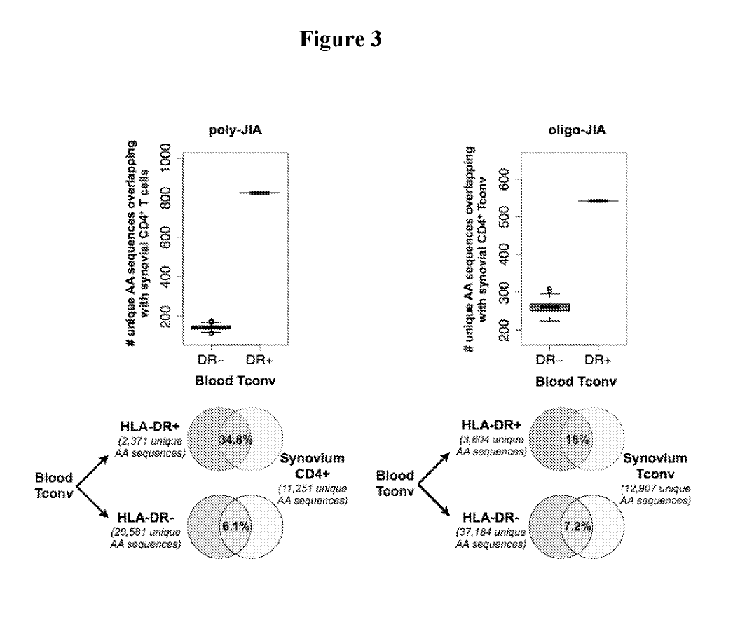

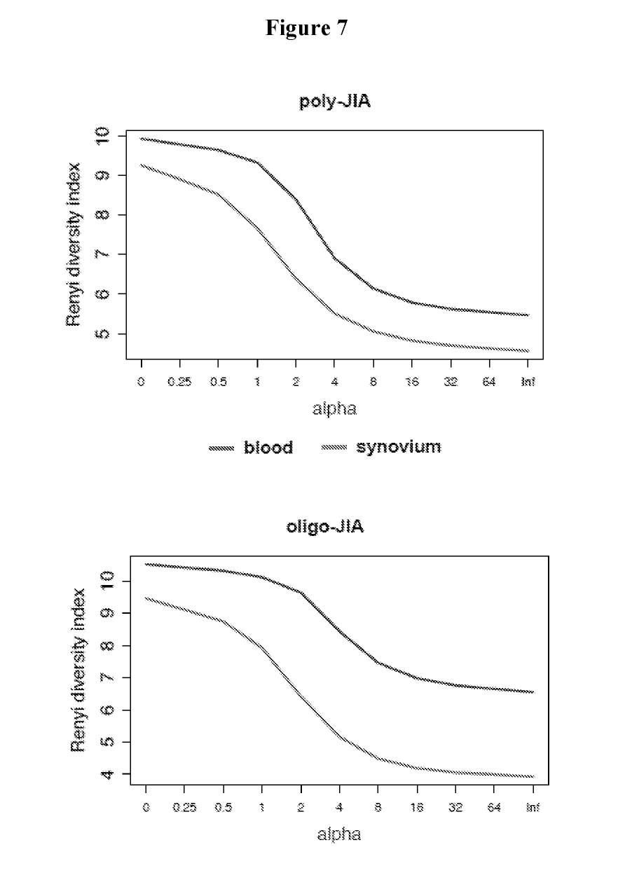

To get insights into the pathogenicity of HLA-DR⁺ T_{conv}, screening with suspected candidate autoantigens was contemplated. However, this is unfeasible with the small amounts of pediatric blood samples (<10 ml). As a practicable alternative, sequencing of TCRβ CDR3 regions was completed on blood and synovial T cells using next-generation sequencing based on the observation that inflamed synovia are highly enriched in arthritogenic T cells. With this method, it was first demonstrated that TCR diversity in the synovium is lower than in blood (FIG. 7), in accordance with previous reports of synovial T cell oligoclonality [Chini et al. (2002) Scand J Immunol 56: 512-517.], and in support of the concept that a limited number of clonotypes is truly pathogenic. Then, the overlap between blood HLA-DR⁺ T_{conv} and synovial T cells was measured. Strikingly, HLA-DR⁺ T_{conv} shared substantially more CDR3 sequences with the synovial TCRs than HLA-DR⁻ T_{conv} (FIG. 3). Based on these data, it is concluded that circulating HLA-DR⁺ T_{conv} are highly enriched in arthritogenic clonotypes contributing to synovial inflammation.

### Example 14

### The Pro-Inflammatory Features of HLA-DR⁺ T. are Exacerbated in Clinical Failures

Having shown that HLA-DR⁺ T_{conv} constitute a reservoir of arthritogenic clonotypes (FIG. 3) expanded in non-responders (FIG. 1), it was considered whether the size of this arthritogenic population is the only factor correlating with responsiveness to therapy, or rather whether there are also intrinsic differences in the pathogenic potential of HLA-DR⁺ T_{conv} between ID and NO ID patients.

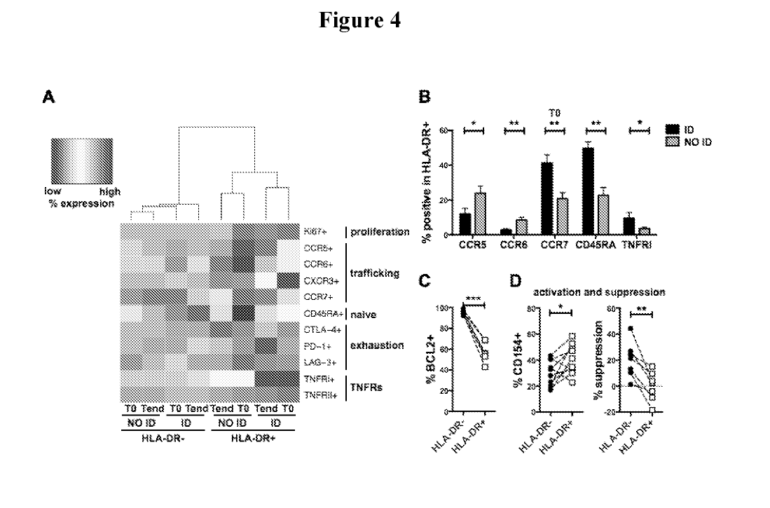

To this aim, the phenotypic characterization reported in FIG. 1 was extended to ID patients and across time, feeding results to unsupervised clustering. The algorithm primarily partitioned samples into HLA-DR⁺ and HLA-DR⁻ T_{conv}; then by responsiveness to therapy, but only within the HLA-DR⁺ population; and finally by time (FIG. 4A). This clustering hierarchy, with time ranking last, suggests that the proinflammatory features of HLA-DR+ T_{conv} remain remarkably stable over the course of the treatment. Moreover, while differences between ID and NO ID patients were secondary to those between HLA-DR⁺ and HLA-DR⁻ T_{conv}, they were sufficient to segregate patients based on responsiveness to therapy. Indeed, a larger proportion of HLA-DR⁺ T_{conv} was positive for the pro-inflammatory chemokine receptors CCR5 and CCR6 in NO ID compared to ID patients at baseline was observed, while the opposite was true for CCR7 and CD45RA, indicating a more aggressive effector phenotype (FIG. 4B). In addition, fewer HLA-DR⁺ T_{conv} expressed TNFRI in NO ID patients. This may negatively influence their susceptibility to TNF-α blockade, potentially contributing to the differential responsiveness to anti-TNF combination therapy. All these differences disappeared after treatment, indicating that the therapy was able to mitigate the hyper-inflammatory features of HLA-DR⁺ T_{conv} observed in NO ID patients at baseline. Of note, the clustering algorithm could not segregate patients by clinical activity within the HLA-DR⁻ subset (FIG. 4A), indicating that the majority of T_{conv} (˜98% HLA-DR⁻) have a negligible role in responsiveness to therapy, in contrast to the tiny fraction (˜2%) of HLA-DR⁺ T cells, which correlate with disease activity.

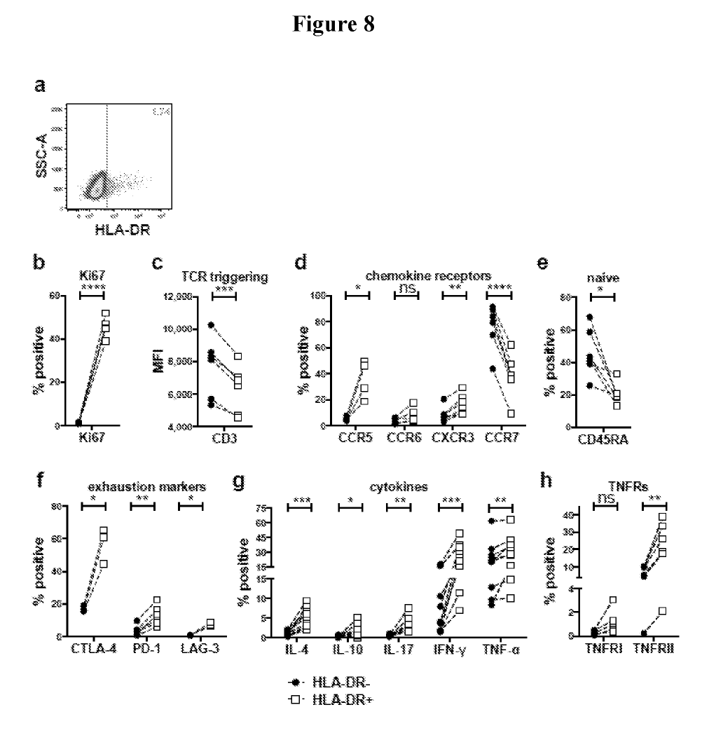

Primed self-reactive, potentially pathogenic T cells are present even in HD, where they are systematically kept under control by mechanisms of peripheral tolerance [Allan et al. (2007) International immunology 19: 345-354]. Consistently, the pro-inflammatory profile of HLA-DR⁺ T_{conv} found in patients was broadly paralleled in HD (FIG. 8). Indeed, HLA-DR⁺ T_{conv} comprised a lower proportion of naive (CCR7⁺ and CD45RA⁺) cells, but more proliferating (Ki67⁺) and highly activated pro-inflammatory T cells. They also showed an across-the-board increase in cytokine production Finally, the fraction of cells positive for TNFRs was larger in HLA-DR⁺ than in HLA-DR⁻ T_{conv}.

The presence of HLA-DR⁺ T cells in HD provided the opportunity to further explore their physiology with fewer constraints on sample amount. First, HLA-DR⁺ T_{conv} displayed higher tendency to apoptosis, as suggested by the lower expression of the antiapoptotic protein BCL2 compared to HLA-DR⁻ T_{conv} (FIG. 4C), and consistent with their late activation stage (FIG. 8). Then, the selective expansion of HLA-DR⁺ T_{conv} in non-responders was investigated (FIG. 1) to determine if the expansion might be due to an intrinsic resistance to T_{reg} cell-mediated suppression. In line with this hypothesis, HLA-DR⁺ Tconv were more responsive to anti-CD3/CD28 stimulation (FIG. 4D, left) and significantly less susceptible to suppression (FIG. 4D, right) than their HLA-DR⁻ counterparts.

Collectively, these data indicate that HLA-DR⁺ T_{conv} are intrinsically hyper-activated and resistant to T_{reg} cell-mediated suppression, and that their pro-inflammatory features are exacerbated in NO ID patients.

### Example 15

### HLA-D12⁺ T_{reg} Cells are Bona Fide Activated T_{reg} Cells

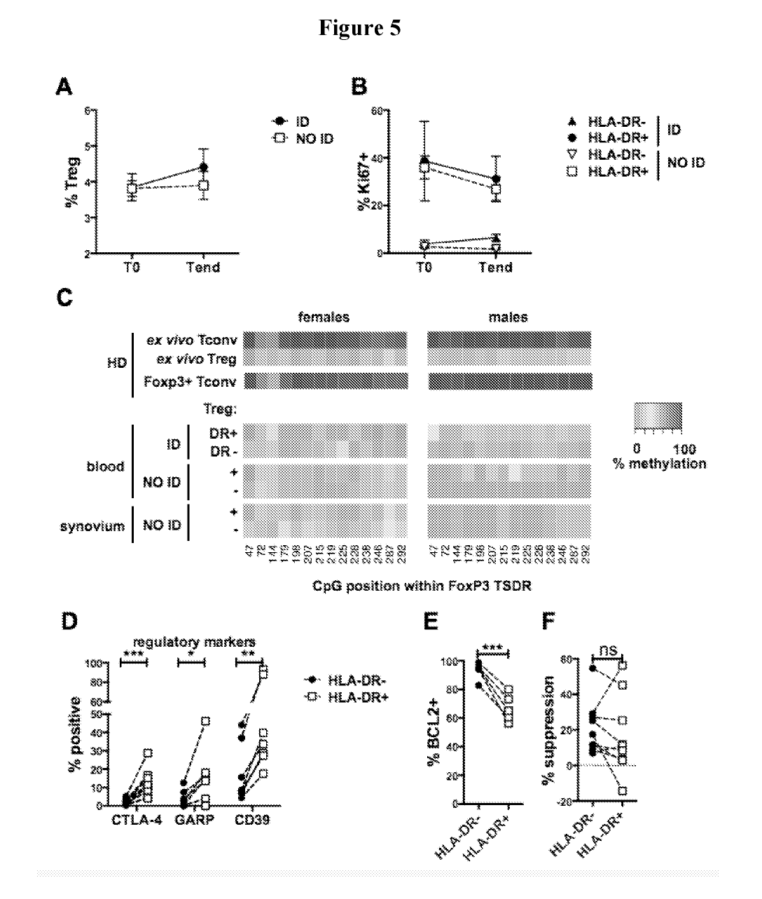

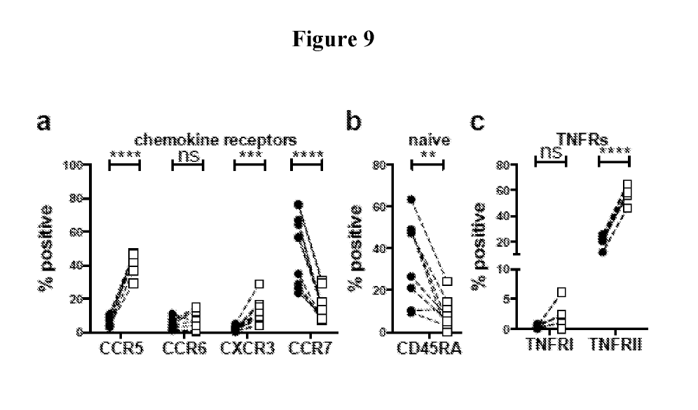

HLA-DR⁺ T_{reg} cells tend to decrease in ID and increase in NO ID patients over the course of the treatment (FIG. 1B); by contrast, total T_{reg} cells remained stable (FIG. 5A). The simultaneous expansion of both HLA-DR⁺ T_{conv} and HLA-DR⁺T_{reg} cells points to an effort of activated HLA-DR⁺ T_{reg} cells to dampen joint inflammation. Consistently, a large proportion of HLA-DR⁺ T_{reg} cells was found positive for the proliferation marker Ki67 (FIG. 5B). Disease activity and time point had no significant impact on Ki67 expression, in line with the observations on HLA-DR⁺ T_{conv} (FIG. 4). HLA-DR⁺ T_{reg} cells were enriched in memory cells programmed to migrate through inflamed tissues and expressing higher proportions of TNFRs compared to HLA-DR⁻ T_{reg} cells (FIG. 9), again mirroring the findings on HLA-DR⁺ T_{conv} (FIG. 2A).

The clinical outcome of non-responders indicates that regulatory mechanisms must have failed, despite the increased number of activated T_{reg} cells. To determine the reasons of this failure, HLA-DR⁺ T_{reg} cell commitment to the regulatory lineage was first investigated by analyzing the methylation profile of the T_{reg} cell-specific demethylated region (TSDR) within the FOXP3 locus [Baron et al. (2007)]. Irrespective of clinical activity, both HLA-DR⁺ and HLA-DR⁻ T_{reg} cells were demethylated in their TSDR, indicating stability of their regulatory phenotype. Moreover, the TSDR methylation profile remained unaffected in the inflammatory environment of the synovium (FIG. 5C). The TSDR methylation results rule out that: a) any failure of T_{reg} cells to restrain inflammation may be due to lineage plasticity; and that b) the cells identified as HLA-DR⁺ T_{reg} cells might be TSDR-methylated T_{conv} transitorily expressing FOXP3 upon activation [Allan et al. (2007) International immunology 19: 345-354].

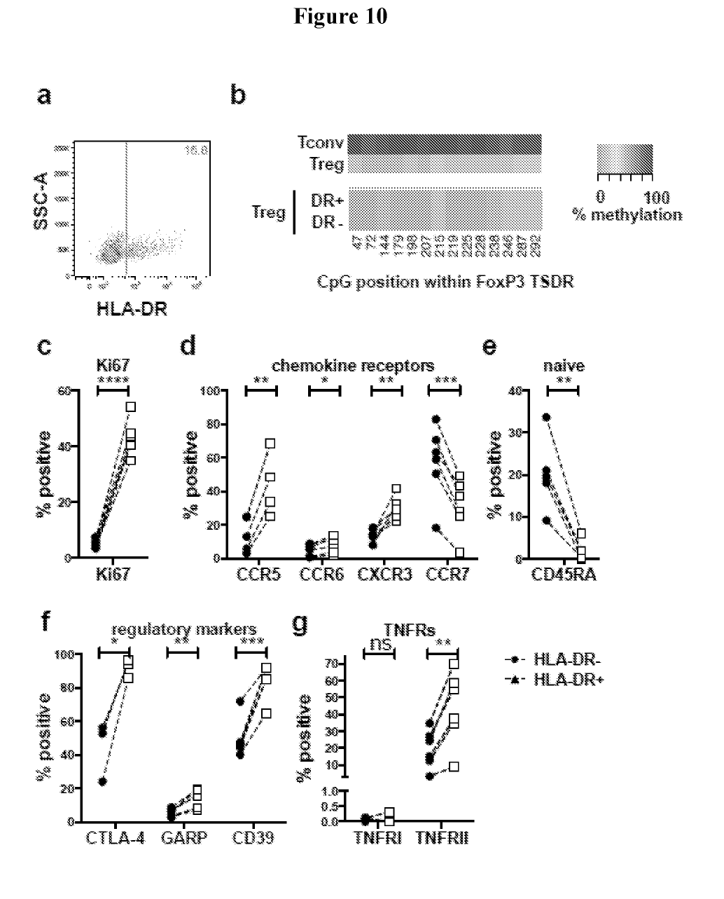

A second explanation for the inability of HLA-DR⁺ T_{reg} cells to control inflammation may be that this subset is intrinsically dysfunctional, even if committed to the regulatory lineage. However, HLA-DR⁺ T_{reg} cells expressed higher levels of CTLA-4, GARP and CD39 compared to HLA-DR⁻ T_{reg} cells (FIG. 5D), demonstrating that HLA-DR⁺ T_{reg} cells are able to upregulate T_{reg} cell-specific effector molecules upon activation. Due to sample amount restrictions, the actual suppressive potential of HLA-DR⁺ T_{reg} cells was addressed by taking advantage of the presence of HLA-DR⁺ T_{reg} cells in HD. Indeed, HLA-DR⁺ T_{reg} cells from HD recapitulated the phenotype and TSDR demethylation of their patient counterparts (FIG. 10). Similarly to T_{conv}, HLA-DR⁺ T_{reg} cells displayed more propensity to apoptosis than HLA-DR⁻ cells (FIG. 5E). HLA-DR⁺ T_{reg} cells were able to suppress T_{conv} activation as efficiently as HLA-DR⁻ T_{reg} cells (FIG. 5F), suggesting that they are not intrinsically dysfunctional.

### Example 16

### HLA-DR Does Not Endow T Cells With Antigen-Presenting Capabilities

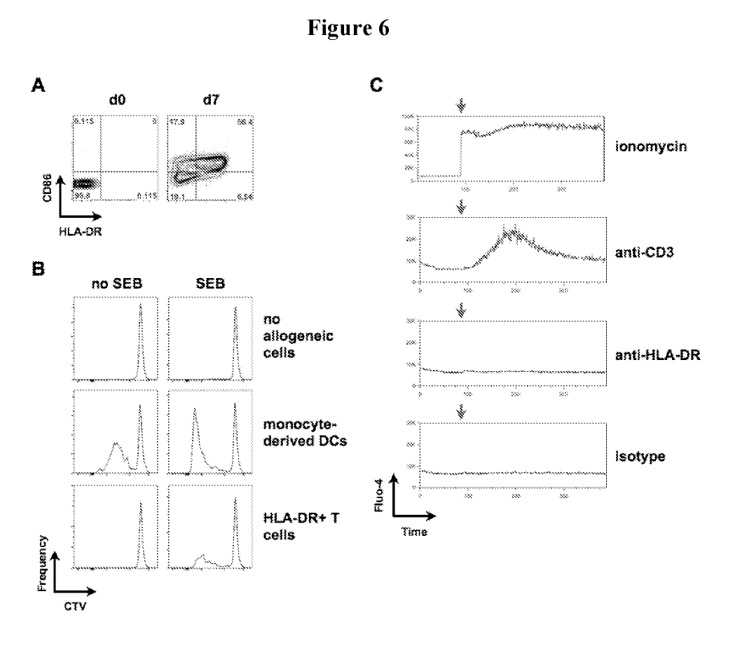

HLA-DR may endow T cells with antigen-presenting capabilities. HLA-DR⁻ T cells were polyclonally stimulated to induce HLA-DR expression. Of note, T cells also up-regulated CD86, suggesting that activated T cells might have both antigen-presenting and co-stimulatory capacity (FIG. 6A). However, only mature HLA-DR⁺ monocyte-derived dendritic cells, but not HLA-DR⁺ T cells, were able to induce proliferation of fresh allogeneic CD4⁺ T cells (FIG. 6B). To exclude that these negative results were due to loss of HLA-DR expression on T cells during the assay, Staphylococcal Enterotoxin B (SEB) was added to the co-culture. SEB bridges specific TCR Vβ domains to MHC class II molecules, leading to T cell activation [Li et al. (1999) Annual review of immunology 17: 435-466. 42]. In the presence of SEB, HLA-DR⁺ T cells were able to stimulate fresh responder T cells, confirming that HLA-DR was still expressed during the assay.

Another possible role for HLA-DR expression on T cells might be back-signaling, which would elicit calcium flux [Odum et al. (1991). Eur J Immunol 21: 123-129]. However, no calcium flux was detected upon crosslinking of HLA-DR on T cells (FIG. 6C).

Based on these data, it was concluded that HLA-DR neither endows T cells with antigen presenting capabilities nor exhibits back-signaling properties.

In sum, a population of arthritogenic HLA-DR⁺ T cells escaping from the site of autoimmune reaction was identified, and expanded in the peripheral blood of clinical failures to anti-TNF combination therapy. These cells represent a circulating, easily accessible reservoir of pathogenic cells in an autoimmune disease, which can be used as described herein to substantially advance the understanding of aberrant self-recognition as well as to design targeted diagnostic tools.

The claim that HLA-DR⁺ T_{conv} are enriched in arthritogenic cells recirculating from the synovium was supported by their functional markers and, most importantly, their TCR repertoire. Indeed, HLA-DR⁺ T_{conv} are enriched in proliferating Ki67⁺ cells and preferentially express pro-inflammatory chemokine receptors and exhaustion markers in the absence of overt infection. In addition, HLA-DR⁺ T_{conv} display lower levels of CD3 compared to HLA-DR⁻ T_{conv}, a sign of TCR-mediated activation, in contrast to cytokine mediated bystander activation [Bangs et al. (2009)]. Moreover, the low expression of BCL2, a key player in the intrinsic apoptotic pathway [Youle and Strasser (2008) Nat Rev Mol Cell Biol 9: 47-59], is a further indication that HLA-DR⁺ T_{conv} may be antigen-activated; indeed, unlike activated bystanders, TCR-triggered T cells preferentially undergo apoptosis using the intrinsic pathway [Bangs et al. (2009)]. However, the most direct evidence of HLA-DR⁺ T_{conv} pathogenicity comes from the analysis of their TCR repertoire: indeed, non-responder HLA-DR⁺ T_{conv} share a consistent fraction of their TCR repertoire with the synovium, where arthritogenic T cells reside; this finding indicates that HLA-DR⁺ T_{conv} comprise pathogenic—and likely autoreactive—cells. Importantly, the highly innovative strategy of leveraging next-generation sequencing of TCRs allowed pinpointing a subset of pathogenic T cells without a priori knowledge of autoantigens, bypassing a limitation that has often plagued advancements in the field.

Interestingly, HLA-DR⁺ T_{reg} cells share phenotypic features with HLA-DR⁺ T_{conv} and are similarly expanded in non-responders, suggesting that HLA-DR⁺ T_{reg} cells may comprise autoreactive clonotypes as well. Indeed, the increased number of arthritogenic Tconv might lead to a compensatory increase of activated T_{reg} cells, in a failed attempt to control inflammation and tissue damage. T_{reg} cells are thought to have higher propensity to recognize self-antigens than T_{conv} [Hsieh et al. (2004) Immunity 21: 267-277.]; consequently, a higher fraction of T_{reg} cells should be activated at any given time, when compared to T_{conv}. In line with this prediction, the percentage of T cells positive for HLA-DR is 10 times higher in T_{reg} cells (10-15%) than in T_{conv} (1-2%), further strengthening the link between HLA-DR expression and potential autoreactivity.

Although HLA-DR⁺ T_{conv} are present in ID patients and HD, consistent with a basal level of self-reactive clonotypes even in the absence of disease, they are selectively expanded in NO ID patients, providing a correlate and suggesting a potential mechanism of unresponsiveness to therapy. Indeed, the increase in HLA-DR⁺ T_{conv} likely results from the expansion of arthritogenic suppression- and therapy-resistant T cells in the synovium, which boost synovial inflammation and thus foster clinical unresponsiveness. By contrast, other hyper-inflammatory features of HLA-DR⁺ T_{conv}, such as increased expression of pro-inflammatory chemokine receptors and reduced expression of TNFRI in NO ID relative to ID patients, seem to play only a minor role, as these anomalies are successfully corrected by treatment but do not translate into clinical benefit. Similarly, intrinsic T_{reg} cell dysfunctonality does not seem to play a major role in driving clinical unresponsiveness. Indeed, although the relative abundance of HLA-DR⁺ T_{reg} cells does correlate with clinical activity, the data shows that HLA-DR⁺ T_{reg} cells are suppressive. However, that extrinsic factors, such as the cytokine milieu in the inflamed joint, may contribute to activated HLA-DR⁺ T_{reg} cell inability to suppress in vivo cannot be excluded.

HLA-DR is not expressed by murine T cells, hindering investigation in vivo. However, the presence of a basal level of HLA-DR⁺ T cells in HD enables the study of the functional role of HLA-DR in CD4⁺ T cells with fewer constraints than in patients. Recently, it was shown that T cells can load self-antigens on their MHCs [Costantino et al. (2012) PLoS One 7: e29805]. In addition, outside-in signaling was reported for MHC class II, despite their short cytoplasmic tails, with a study reporting Ca²⁺ mobilization after HLA-DR cross-linking on T cell clones [Li et al. (1999)]. However, in the current experimental system, HLA-DR cross-linking in CD4⁺ T cells did not result in any meaningful biological read-out.

Although HLA-DR has been known as a T cell activation marker [Evans et al. (1978) J Exp Med 148: 1440-1445.], it has been used to track antigen-specific clonotypes only for blood-borne infectious diseases, such as HIV [Orendi et al. (1998) J Infect Dis 178: 1279-1287.]. Herein, it has been proposed for the first time to use HLA-DR for detecting pathogenic T cells recirculating from peripheral tissues. There are three major advancements connected thereto. First, HLA-DR can be used to define pathogenic clonotypes without prior knowledge of antigen specificity, a major unmet medical need in autoimmunity. Indeed, this is a crucial limitation differentiating infectious diseases such as HIV, where microbial antigens are known, from autoimmune diseases such as JIA, where most antigens have not been identified. HLA-DR also bypasses another limitation imposed by tetramer-based strategies or restimulation approaches, namely the ability to detect only high-affinity TCRs. Second, HLA-DR is not downregulated when pathogenic T cells, activated in peripheral tissues such as the synovium, recirculate into the bloodstream. Again, this is a key difference between blood-borne diseases such as HIV, where it is easy to envision that pathogenic T cells can be easily isolated from blood, from autoimmune diseases such as JIA, where uncovering a reservoir of circulating pathogenic cells is unexpected. The finding that HLA-DR provides an easy and minimally-invasive access to the few recirculating pathogenic T cells is especially useful for autoimmune diseases with difficult or no access to the actual site of inflammation, such as multiple sclerosis. In addition, the long-lasting expression window of HLA-DR allows the capture of a wide fraction of T cell emigrants from peripheral tissues, which would instead down-regulate early activation markers such as CD69 and CD40L by the time they reach the blood. Finally, the seeming lack of signalling properties of HLA-DR in T cells is advantageous insofar as it allows the isolation of unperturbed and viable T cells, differently from other surrogate markers of antigen encounter (e.g. Ki67). The isolation of pathogenic T cells through HLA-DR followed by screenings with candidate self-peptides in non-pediatric settings characterized by relaxed sample constraints, such as adult rheumatoid arthritis, may facilitate the definition of their TCR specificity, and could be a novel platform to discover yet unknown autoantigens.

### Example 17

### Unfractionated Circulating T Cells From JIA Patients With Active Disease Do Not Reflect Synovial Inflammation

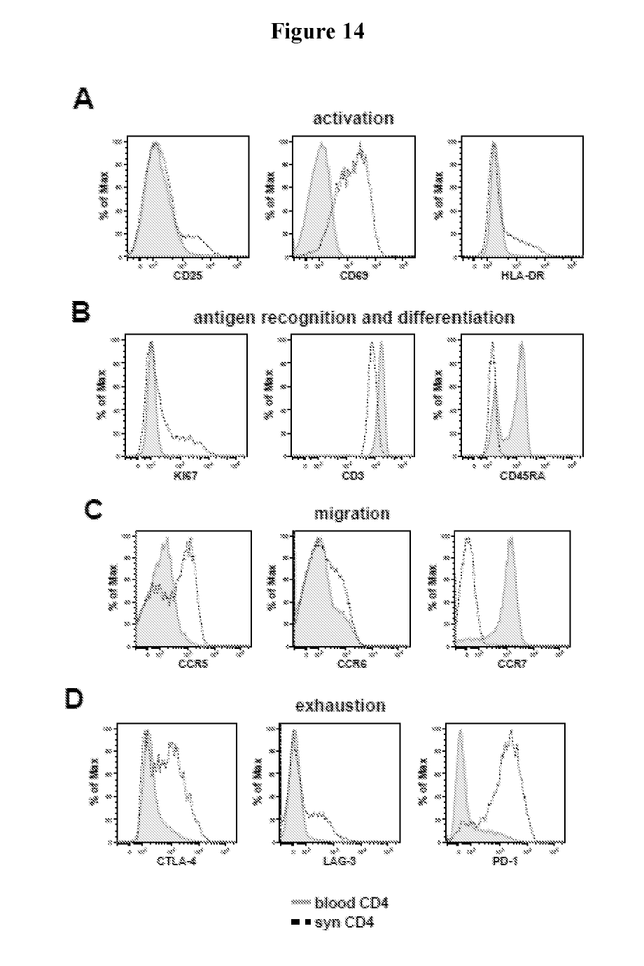

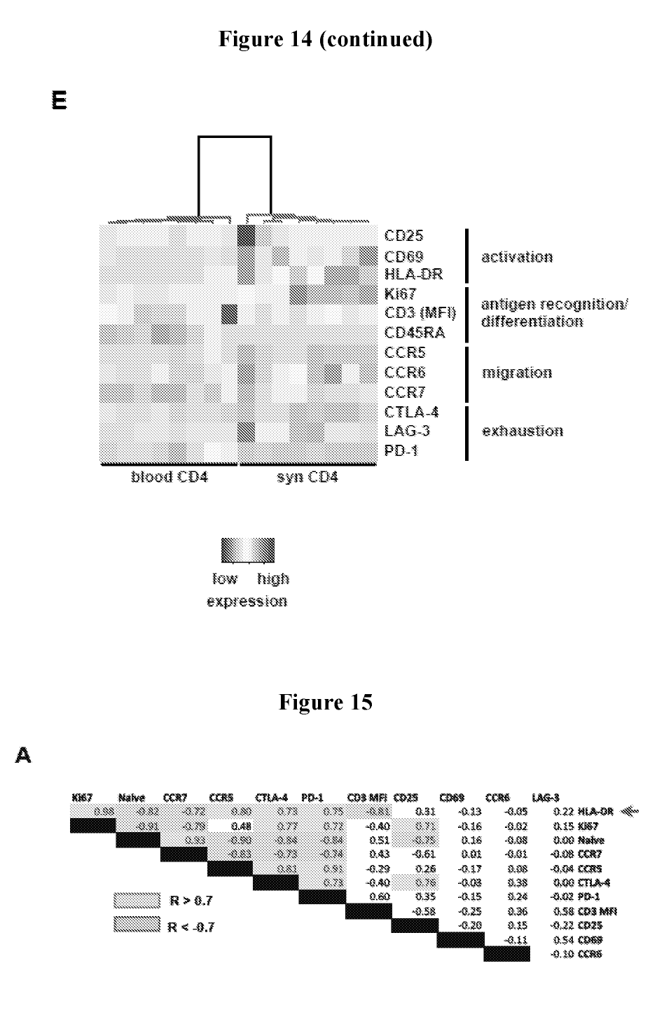

Circulating immune cells cannot faithfully represent the situation found in peripheral inflamed tissues, which are enriched in pathogenic T cells. The scientific community is more and more aware of this issue and is thus moving away from immunological studies in the blood to embrace tissue immunology. FIG. 14A exemplifies this concept with blood and synovial samples from juvenile idiopathic arthritis (JIA) patients. Synovial T cells mainly comprised highly activated CD69+CD45RA− memory T cells, and were enriched in CD25⁺ and HLA-DR⁺ cells, while circulating T cells showed a mixed naive/memory resting phenotype (FIG. 14A-B). Consistent with recent antigen recognition, a large fraction of synovial T cells expressed Ki67 (a sign of in vivo proliferation) and downregulated CD3 (FIG. 14B). Moreover, they preferentially expressed the pro-inflammatory chemokine receptors CCR5 and CCR6 over the lymphoid-homing CCR7 (FIG. 14C). Finally, synovial T cells expressed high levels of exhaustion markers, such as CTLA-4, LAG-3 and PD-1, again consistent with prolonged antigen exposure (FIG. 14D). Importantly, unsupervised clustering of the phenotypic data clearly depicted the dissimilarity between blood and synovium signatures, as the algorithm perfectly segregated the samples based on tissue of origin (FIG. 14E).

Altogether, these data support the concept that unfractionated blood is a limited tool for any investigation aiming at brake-through discoveries in peripherally localized diseases, and that a much finer dissection is needed for tackling complex pathogenic mechanisms.

### Example 18

### HLA-DR is a Good Candidate For Isolating Synovial-Like T Cells From Peripheral Blood

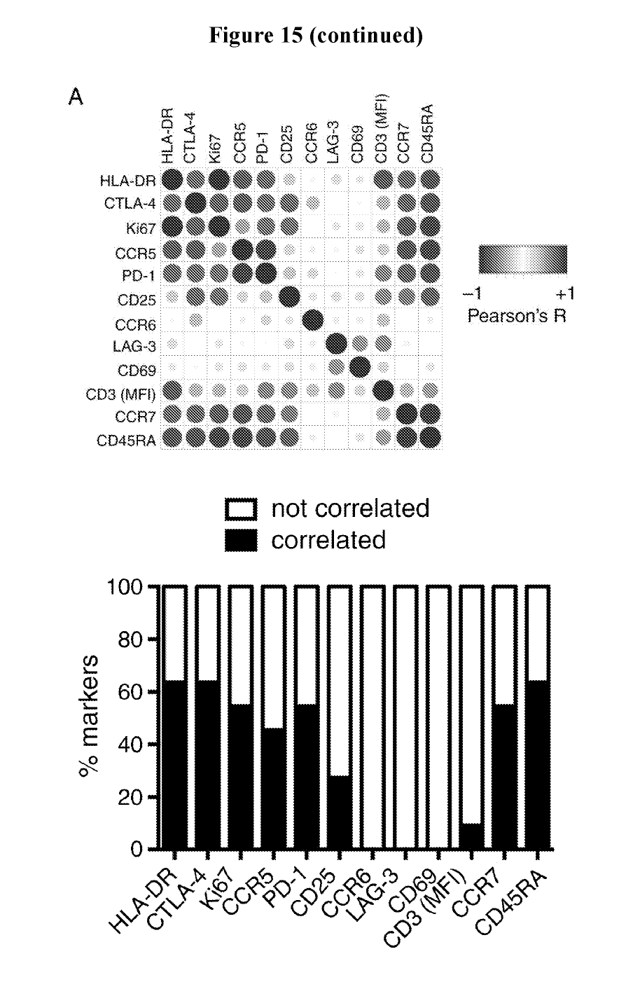

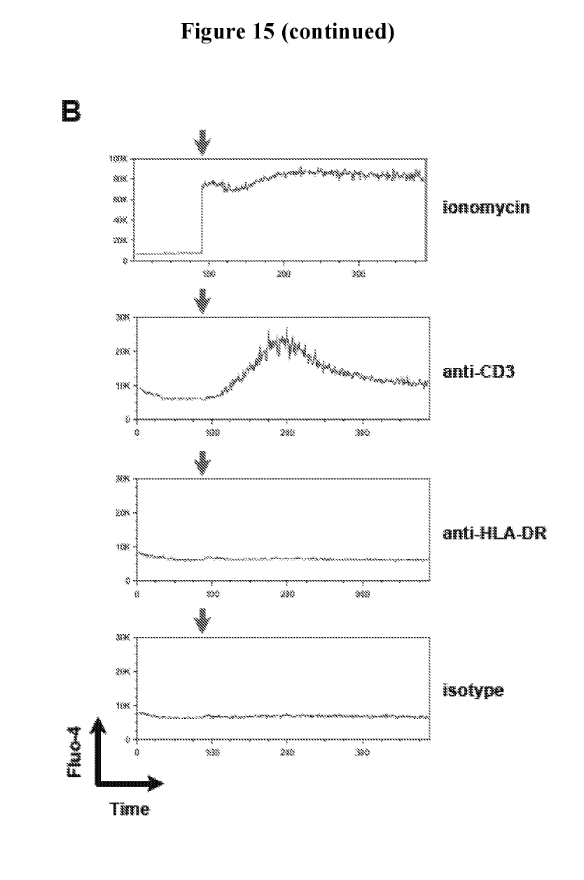

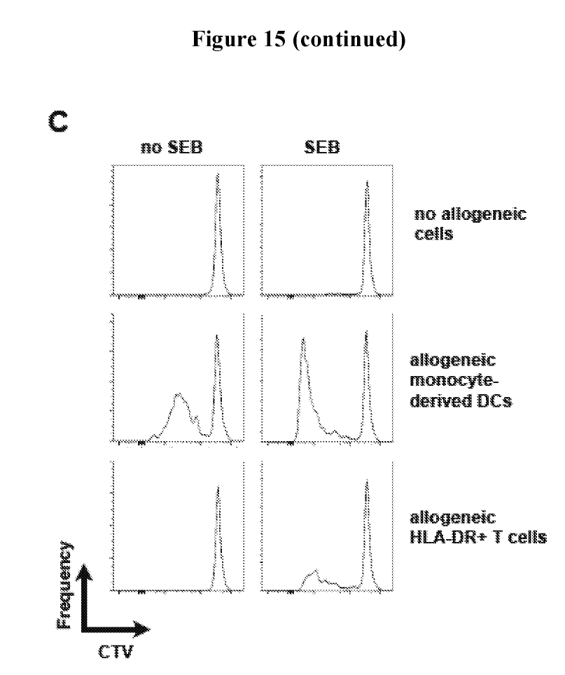

If any fraction of pathogenic T cells escape the inflamed tissue, such population would have a profile similar to that of synovial T cells, enriched in arthritogenic T cells. Therefore, to identify a suitable marker for recirculating pathogenic T cells, the molecules that highly represented in the synovium were focused on (FIG. 14). Interestingly, most of them were highly intercorrelated in peripheral CD4⁺ T cells, indicating that the same population might be pinpointed by any of these proteins in the circulation. Some notable exceptions were CD69 and LAG-3, which very poorly correlated with any other molecule, and CD25, which correlated with some but not all (FIG. 15A). These data suggest that different T cell activation markers have unique features; as such, they cannot be interchangeably used as correlates for peripheral inflammation.

Among the highly correlated antigens, HLA-DR was particularly appealing as sorting marker for the following reasons: 1) it is a surface protein, allowing sorting of living cells and subsequent functional analyses, in contrast to otherwise relevant intracellular antigens requiring fixation (Ki67 and CTLA-4); 2) it does not seem to have a defined signaling capability in T cells, allowing sorting of unperturbed T cells. Several studies were performed on the last topic, but results were conflicting and inconclusive. No calcium flux upon cross-linking of HLA-DR+ T cell lines was detected (FIG. 15B), which demonstrates that HLA-DR does not exhibit any back-signaling activity. Another possible role for HLA-DR expression on T cells would be to endow them with antigen-presenting capabilities. Only mature HLA-DR⁺ monocyte-derived dendritic cells, but not HLA-DR⁺ T cell lines, were able to induce proliferation of fresh allogeneic CD4⁺ T cells (FIG. 2C). To rule out that these negative results were due to HLA-DR downregulation on T cells during the assay, Staphylococcal Enterotoxin B (SEB) was added to the co-culture. SEB bridges specific TCR Vβ domains to MHC class II molecules, leading to T cell activation. In the presence of SEB, HLA-DR⁺ T cell lines were able to stimulate fresh responder T cells, confirming that HLA-DR was still expressed during the assay.

Therefore, HLA-DR can be used to sort viable and unperturbed T cells potentially similar to synovial T cells.

### Example 19

### Circulating Pathogenic-Like T Cells From Patients With Active Disease Mirror the Inflammatory Synovial T Cell Signature

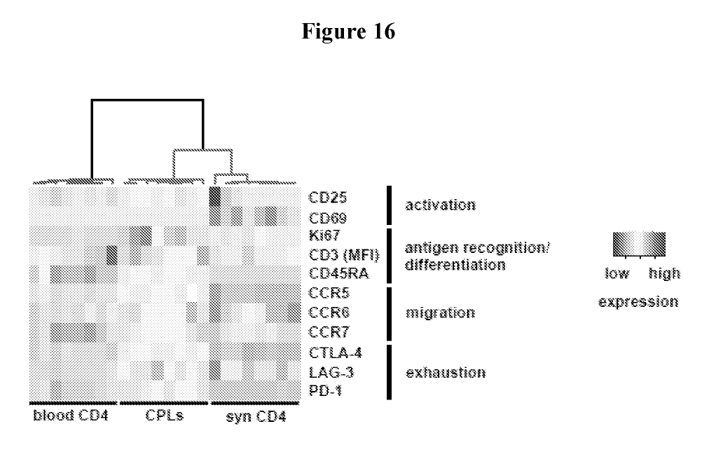

HLA-DR was tested to determine if it is indeed a marker of pathogenic-like T cells in the circulation. Blood HLA-DR⁺ T cells were remarkably different from the bulk of blood CD4⁺HLA-DR T cells (FIG. 16). Indeed, these circulating pathogenic-like lymphocytes (CPLs) were enriched in activated, memory-like, antigen-experienced Ki67⁺ cells. Importantly, they might have been exposed to autoantigens in affected joints, as they preferentially expressed CCR5 and CCR6 over CCR7. Finally, CPLs expressed high levels of exhaustion markers. Strikingly, the unsupervised clustering algorithm segregated CPLs apart from blood T cells, but together with synovial T cells (FIG. 16), based on their phenotypic characteristics, demonstrating that they closely mirror the synovial T cell signature.

In summary, CPLs represent a small population of synovial-like, antigen-experienced T cells able to recirculate through inflamed sites.

### Example 20

### CPLs are Enriched in Clonotypes of Synovial T Cells

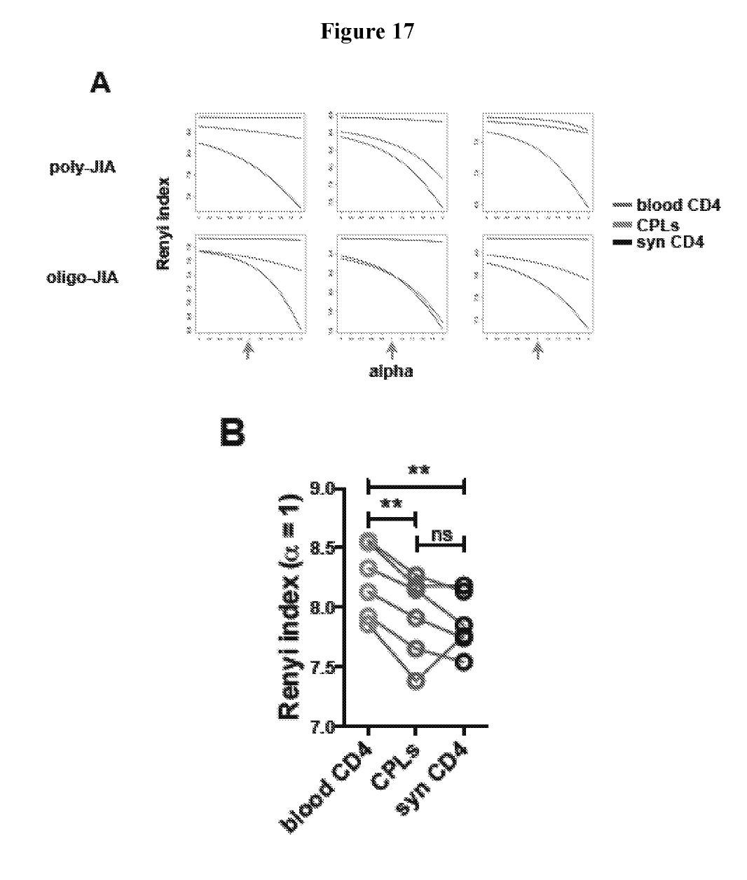

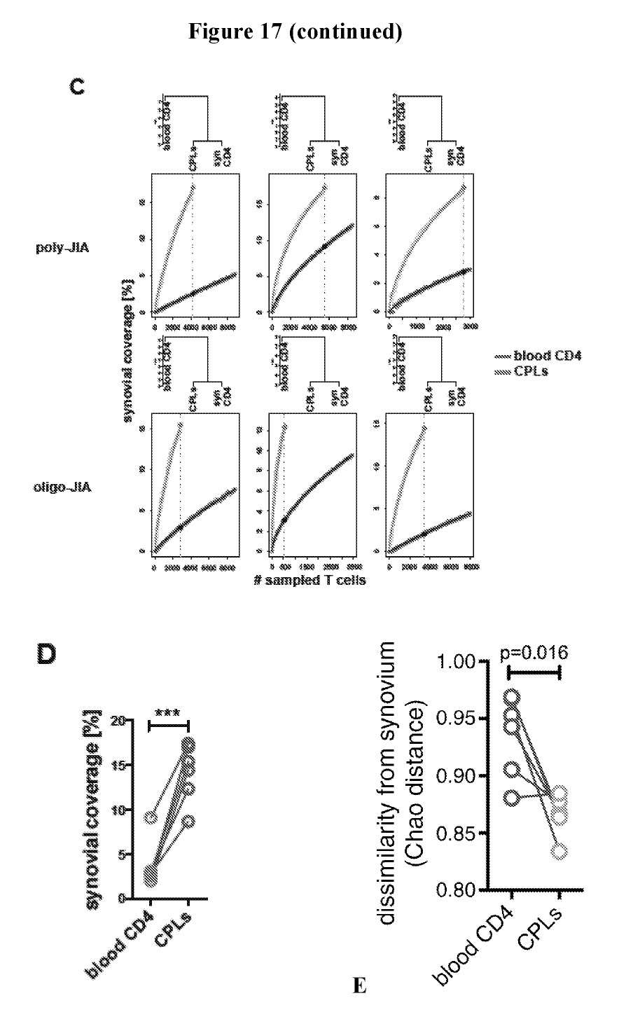

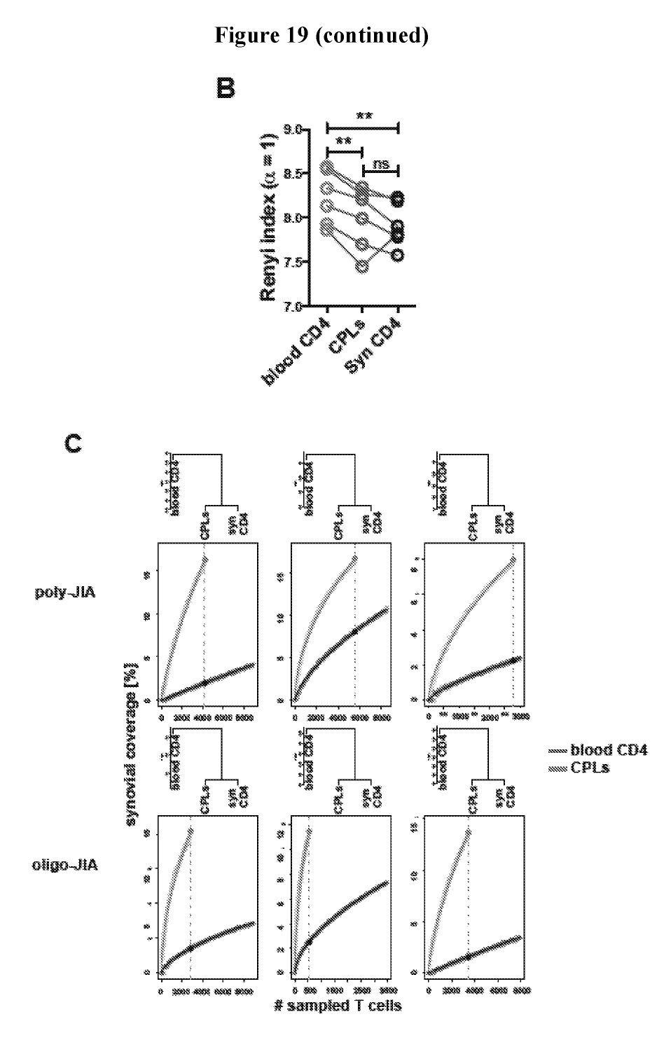

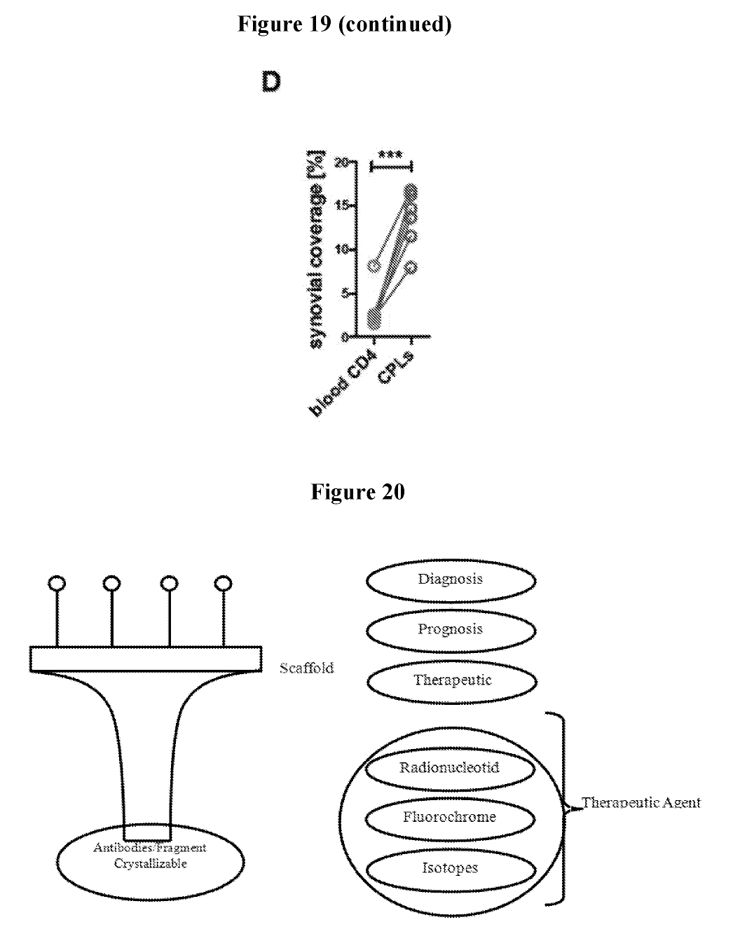

To get formal proof of CPL pathogenicity, a screening with candidate autoantigens was contemplated. However, this is unfeasible with the small amounts of pediatric blood samples (<10 ml). In addition, only a few autoantigens are known in JIA, which would lead to an overwhelmingly high rate of false negative results. As a practicable—and antigen-agnostic—alternative, we took advantage of the observation that inflamed synovia are highly enriched in arthritogenic T cells. Therefore, the TCR repertoires of blood and synovial T cells were compared through next-generation sequencing of TCRβ CDR3 regions, the main determinants of TCR specificity. In accordance with previous reports of synovial T cell oligoclonality, the TCR diversity in the synovium was much lower than in blood. Importantly, also CPLs were oligoclonal when compared with the bulk of blood T cells, and in some instances as diverse or even less diverse than the synovium, at both amino acid and nucleotide levels (FIG. 17A-B and FIG. 19A-B, respectively). These data further support the concept that a limited number of clonotypes is truly pathogenic.

Then, CPL and synovial T cell repertoires were measured and compared. Upon clustering analysis based on TCR repertoire distance, CPLs segregated together with synovial T cells and apart from blood T cells (FIG. 17C and FIG. 19C, top panels). Moreover, CPLs shared a substantially higher fraction of CDR3 sequences with synovial TCRs than blood T cells (FIG. 17C-D and FIG. 19C-D, respectively). As evident from the observed trend, the overlap increases as a function of the number of sequenced T cells. Therefore, bigger blood samples would yield even higher TCR coverage. Altogether, these data demonstrate that CPLs are highly enriched in clonotypes contributing to synovial inflammation.

### Example 21

### CPLs Correlate With Unresponsiveness to Therapy and Disease Activity in Both Juvenile and Adult Autoimmune Arthritis

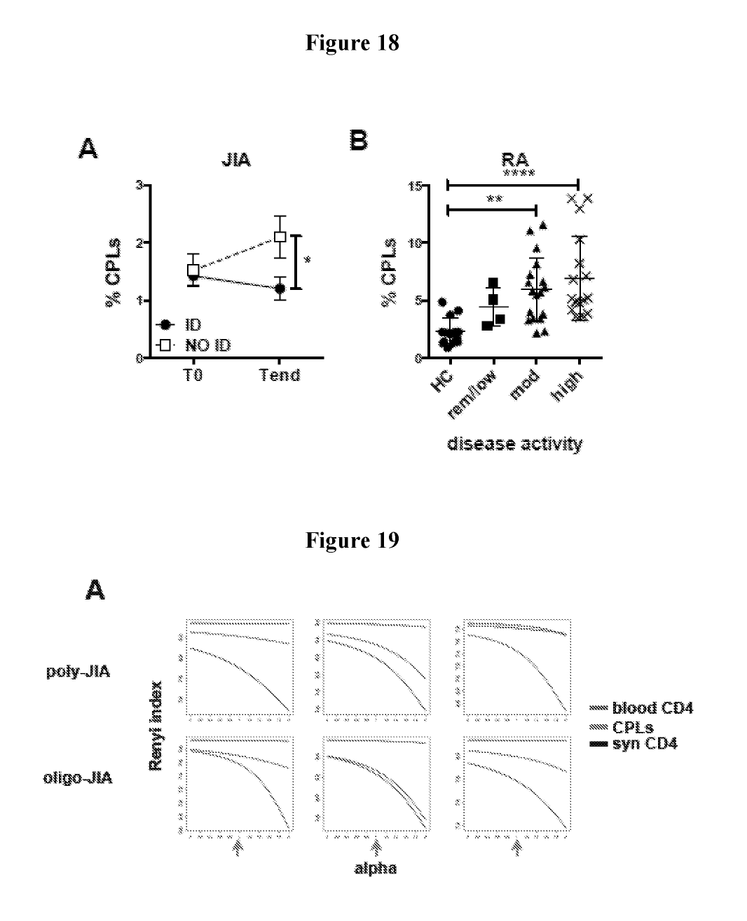

Finally, the clinical relevance was investigated. Given their enrichment in pathogenic clonotypes, it was hypothesized that CPLs would be increased in patients with active disease as compared to patients reaching inactive disease (ID). This hypothesis was tested on peripheral blood samples from JIA patients, collected before (T0) and after (Tend) therapy, and stratified at Tend based on whether they reached ID or not (NO ID). CPLs slightly declined in prospective ID patients, but substantially increased in NO ID patients, resulting in a 2-fold difference at Tend (FIG. 18A). Thus, CPL increase mirrors the increased synovial inflammation clinically observed in NO ID patients. Although the results were clear-cut in JIA, it was investigated if they might be extended to other autoimmune diseases. Therefore, the presence of CPLs was examined for adult rheumatoid arthritis (RA). Importantly, CPLs were expanded in RA patients as compared to healthy controls, and substantially increased with worsening activity score even in adult RA (FIG. 18B), conferring a broader significance of the results beyond pediatric autoimmunity.

1. A method of determining the effectiveness of a therapeutic regimen in a patient afflicted by an autoimmune disease, comprising:
a) obtaining a biological sample from the patient;
b) enriching CD4⁺ T cells expressing HLA-DR from the biological sample;
c) optionally expanding the CD4⁺ T cells expressing HLA-DR; and
d) determining the number of CD4⁺ T cells expressing HLA-DR, wherein an elevated number of CD4⁺T cells expressing HLA-DR compared to a reference value indicates an increased likelihood that the patient is or will be failing therapy.

2. The method of claim 1, wherein the CD4⁺ T cells expressing HLA-DR are conventional T cells or regulatory T cells.

3. The method of claim 1, wherein the CD4⁺ T cells expressing HLA-DR are HLA-DR⁺CD14⁻CD4⁺CD25^{low/-} conventional T cells, comprising HLA-DR⁺CD14CD4⁺CD25^{low/-} FoxP3 conventional T cells or the CD4⁺ T cells expressing HLA-DR are HLA-DR⁺CD14⁻CD3⁺CD4⁺CD25⁺ regulatory T cells, comprising HLA-DR⁺CD14⁻CD3⁺CD4⁺CD25⁺FoxP3⁺ regulatory T cells.

4. (canceled)

5. The method of claim 1, wherein the biological sample is obtained during the therapeutic regimen and wherein an elevated number of T cells expressing HLA-DR compared to a reference value indicates an increased likelihood that the patient is failing therapy.

6. The method of claim 1, wherein the therapeutic regimen comprises administration of a biological agent, comprising an antibody.

7. (canceled)

8. The method of claim 1, wherein the therapeutic regimen comprises administration of methotrexate and/or prednisolone.

9. The method of claim 1, wherein the biological sample a blood sample.

10. The method of claim 1, wherein the autoimmune disease is selected from rheumatoid arthritis, juvenile idiopathic arthritis and multiple sclerosis.

11. The method of claim 1, further comprising determining a marker expressed by the CD4⁺ T cells expressing HLA-DR, the marker optionally being selected from the group consisting of Ki67, CCR5, CCR6, CTLA-4, LAG-3, PD-1, IL-4, IL-17, TNF-γ, and TNF-a.

12. A method of determining a patient's risk of developing an autoimmune disease, comprising:
a) obtaining a biological sample from the patient;
b) enriching CD4⁺ T cells expressing HLA-DR from the biological sample;
c) optionally expanding the CD4⁺ T cells expressing HLA-DR; and
d) determining the number of CD4⁺ T cells expressing HLA-DR, wherein an elevated number of CD4⁺ T cells expressing HLA-DR compared to a reference value indicates an increased likelihood the patient has or is at risk of developing an autoimmune disease.

13. The method of claim 12, wherein the CD4⁺ T cells expressing HLA-DR are conventional T cells or regulatory T cells, preferably conventional T cells.

14. The method of claim 12, wherein the CD4⁺ T cells expressing HLA-DR are HLA-DR⁺CD14CD4⁺CD25^{low/-} conventional T cells, comprising HLA-DR⁺CD14CD4⁺CD25^{low/-} FoxP3 conventional T cells or the CD4⁺ T cells expressing HLA-DR are HLA-DR⁺CD14⁻CD3⁺CD4⁺CD25⁺ regulatory T cells, comprising HLA-DR⁺CD14⁻CD3⁺CD4⁺CD25⁺FoxP3⁺ regulatory T cells.

15. (canceled)

16. (canceled)

17. The method of claim 12, wherein the autoimmune disease is selected from rheumatoid arthritis, juvenile idiopathic arthritis and multiple sclerosis.

18. The method of claim 12, further comprising determining a marker expressed by the CD4⁺ T cells expressing HLA-DR, the marker optionally being selected from the group consisting of Ki67, CCRS, CCR6, CTLA-4, LAG-3 and PD-1, IL-4, IL-17, TNF-γ, and TNF-a.

19. A method for identifying autoantigens in a patient afflicted by an autoimmune disease, comprising:
a) obtaining a biological sample from the patient;
b) enriching CD4⁺ T cells expressing HLA-DR from the biological sample;
c) contacting the biological sample with a candidate autoantigen; and
d) determining the number of CD4⁺ T cells expressing HLA-DR, wherein an elevated number of CD4⁺ T cells expressing HLA-DR compared to a reference value indicates an increased likelihood that the candidate autoantigen is an autoantigen related to the autoimmune disease.

20. The method of claim 19, wherein the CD4⁺ T cells expressing HLA-DR are conventional T cells or regulatory T cells.

21. The method of claim 19, wherein the CD4⁺ T cells expressing HLA-DR are HLA-DR⁺ CD14 CD4⁺CD25^{low/} conventional T cells, comprising HLA-DR⁺CD14CD4⁺CD25^{low/-} FoxP3 conventional T cells or the CD4⁺T cells expressing HLA-DR are HLA-DR⁺CD14⁻CD3⁺CD4⁺CD25⁺ regulatory T cells, comprising HLA-DR⁺CD14⁻CD3⁺CD4⁺CD25⁺FoxP3 ⁺regulatory T cells.

22. (canceled)

23. (canceled)

24. The method of or claim 19 wherein the autoimmune disease is selected from rheumatoid arthritis, juvenile idiopathic arthritis and multiple sclerosis.

25. The method of claim 19, wherein the candidate autoantigen is isolated from a biological sample that is a synovial sample.

26. The method of claim 19, further comprising determining a marker expressed by the CD4⁺ T cells expressing HLA-DR, the marker optionally being selected from the group consisting of Ki67, CCR5, CCR6, CTLA-4, LAG-3, PD-1, IL-4, IL-17, TNF-γ, and TNF-a.
